SEQUENCE LISTING (30 sequences)

SEQ ID NO: 1 - protein, 174 aa, Human adenovirus 5
AKKRSDQHPVRVRGHYRAPWGAHKRGRTGRTTVDDAIDAVVEEARNYTPTPPPVSTVDAAIQTVVRGARRYAKMKRRRRRVARRHRRRPGTAAQRAAAALLNRARRTGRRAAMRAARRLAAGIVTVPPRSRRRAAAAAAAAISAMTQGRRGNVYWVRDSVSGLRVPVRTRPPRN

SEQ ID NO: 2 - protein, 18 aa, Human adenovirus 5
GGAKKRSDQHPVRVRGHY

SEQ ID NO: 3 - protein, 16 aa, Artificial Sequence
  chymotryptic peptide
AKKRSDQHPVRVRGHY

SEQ ID NO: 4 - protein, 12 aa, Artificial Sequence
  peptide
GHYRAPWGAHKR

SEQ ID NO: 5 - protein, 20 aa, Artificial Sequence
  peptide
GRTGRTTVDDAIDAVVEEAR

SEQ ID NO: 6 - protein, 25 aa, Artificial Sequence
  peptide
NYTPTPPPVSTVDAAIQTVVRGARR

SEQ ID NO: 7 - protein, 21 aa, Artificial Sequence
  peptide
NYTPTPPPVSTVDAAIQTVVR

SEQ ID NO: 8 - protein, 9 aa, Artificial Sequence
  peptide
WVRDSVSGL

SEQ ID NO: 9 - protein, 19 aa, Artificial Sequence
  peptide
FPSKMFGGAKKRSDQHPVR

SEQ ID NO: 10 - protein, 47 aa, Artificial Sequence
  peptide
AKKRSDQHPVRVRGHYRAPWGAHKRGRTGRTTVDDAIDAVVEEARNY

SEQ ID NO: 11 - protein, 10 aa, Artificial Sequence
  peptide
AKKRSDQHPV

SEQ ID NO: 12 - protein, 15 aa, Artificial Sequence
  peptide
AKKRSDQHPVRVRGH

SEQ ID NO: 13 - protein, 20 aa, Artificial Sequence
  peptide
AKKRSDQHPVRVRGHYRAPW

SEQ ID NO: 14 - protein, 25 aa, Artificial Sequence
  peptide
AKKRSDQHPVRVRGHYRAPWGAHKR

SEQ ID NO: 15 - protein, 44 aa, Artificial Sequence
  peptide
HKRGRTGRTTVDDAIDAVVEEARNYTPTPPPVSTVDAAIQTVVR

SEQ ID NO: 16 - DNA, 20 bp, Artificial Sequence
  DBP primer
gccattgcgcccaagaagaa

SEQ ID NO: 17 - DNA, 24 bp, Artificial Sequence
  DBP primer
ctgtccacgattacctctggtgat

SEQ ID NO: 18 - DNA, 19 bp, Artificial Sequence
  protein VII primer
gcgggtattgtcactgtgc

SEQ ID NO: 19 - DNA, 19 bp, Artificial Sequence
  protein VII primer
cacccaatacacgttgccc

SEQ ID NO: 20 - DNA, 21 bp, Artificial Sequence
  cellular tubulin primer
ccagatgccaagtgacaagac

SEQ ID NO: 21 - DNA, 21 bp, Artificial Sequence
  cellular tubulin primer
gagtgagtgacaagagaagcc

SEQ ID NO: 22 - DNA, 22 bp, Artificial Sequence
  HMGB1 primer
taactaaacatgggcaaaggag

SEQ ID NO: 23 - DNA, 20 bp, Artificial Sequence
  HMGB1 primer
tagcagacatggtcttccac

SEQ ID NO: 24 - DNA, 22 bp, Artificial Sequence
  beta actin primer
gcaccacaccttctacaatgag

SEQ ID NO: 25 - DNA, 22 bp, Artificial Sequence
  beta actin primer
ggtctcaaacatgatctgggtc

SEQ ID NO: 26 - protein, 5 aa, Artificial Sequence
  motif
AKKRS

SEQ ID NO: 27 - protein, 16 aa, Artificial Sequence
  peptide
MKWVTFISLLFSSAYS

SEQ ID NO: 28 - protein, 30 aa, Artificial Sequence
  peptide
MDSKGSSQKGSRLLLLLVVSNLLLCQGVVS

SEQ ID NO: 29 - protein, 59 aa, Artificial Sequence
  peptide
MGQIVTMFEALPHIIDEVININVIIVLIIITSIKAVYNFATCGILALVSFLFLAGRSCG

SEQ ID NO: 30 - protein, 98 aa, Artificial Sequence
  peptide
MPNHQSGSPTGSSDLLLSGKKQRPHLALRRKRRREMRKINRKVRRMNLAPIKEKTAWQHLQALISEAEEVLKTSQTPQNSLTLFLALLSVLGPPPVTG

(19) United States
(12) Patent Application Publication
(10) Pub. No.: US 2019/0276499 A1
(43) Pub. Date: Sep. 12, 2019

(54) Protein VII Fragments and Methods of Use Thereof for the Treatment of Inflammatory Disorders

(71) Applicant: The Children's Hospital of Philadelphia, Philadelphia, PA (US)

(72) Inventors: Matthew D. Weitzman, Bala Cynwyd, PA (US); Daphne C. Avgousti, Philadelphia, PA (US); Christin Herrmann, Philadelphia, PA (US); Andrew J. Paris, Bala Cynwyd, PA (US); G. Scott Worthen, Merion Station, PA (US)

(21) Appl. No.: 16/312,010

(22) PCT Filed: Jun. 22, 2017
(86) PCT No.: PCT/US17/38877

§ 371 Date: Dec. 20, 2018

Related U.S. Application Data
(60) Provisional application No. 62/353,345, filed on Jun. 22, 2016.

(51) Int. Cl. C07K 14/075; C12N 15/86; A61K 9/00; A61K 48/00; A61P 11/16; A61P 31/04; A61P 9/00

(52) U.S. Cl. CPC C07K 14/075; C12N 15/86; A61K 9/0073; A61K 38/00; A61P 11/16; A61P 31/04; A61P 9/00; A61K 48/0058

(57) ABSTRACT

Compositions and methods for treating inflammation are disclosed. More specifically, the invention provides biologically active fragments of protein VII from human adenovirus serotypes useful for reducing inflammatory symptoms.

This application claims priority to U.S. Provisional Application No. 62/353,345 filed Jun. 22, 2016, the entire disclosure being incorporated herein by reference as though set forth in full.

### FIELD OF THE INVENTION

The present invention relates to the field of inflammation and related diseases and disorders. More specifically, the invention provides compositions and methods for treating inflammation.

### BACKGROUND OF THE INVENTION

Several publications and patent documents are cited throughout the specification in order to describe the state of the art to which this invention pertains. Full citations of these references can be found throughout the specification. Each of these citations is incorporated herein by reference as though set forth in full.

High mobility group box 1 (HMGB1) is a chromatin organizer protein, ubiquitously expressed in cells. Following a number of stressful events, HMGB1 is released into the cytosol and thence into the extracellular space. HMGB1 is found in the plasma in a variety of diseases including, without limitation, sepsis, trauma, acute respiratory distress syndrome (ARDS), and multi-organ failure. HMGB1 operates as a Danger (or Damage)—Associated Molecular Pattern (DAMP) by interacting with TLR4, RAGE, and other receptors and signaling molecules to promote inflammation and injury. HMGB1, which can be released by activated macrophages, can activate macrophages/monocytes to release proinflammatory cytokines, upregulate endothelial adhesion molecules, and stimulate epithelial cell barrier failure (Wang et al. (2004) J. Intern. Med., 255:320-31). Anti-HMGB1 antibodies have been shown to mitigate the activity of HMGB1 (Wang et al. (2004) J. Intern. Med., 255:320-31). However, improved methods for modulating HMGB1 activity are needed.

### SUMMARY OF THE INVENTION

In accordance with the instant invention, protein VII peptides are provided. In a particular embodiment, the protein VII peptide is less than 80 amino acids in length. In a particular embodiment, the protein VII peptide comprises an N-terminal fragment of protein VII. The protein VII peptides of the instant invention may comprise at least 80%, 90%, 95%, or 100% identity with an adenovirus protein VII. The adenovirus can be any adenovirus, particularly a human adenovirus. Preferred adenovirus serotypes include adenovirus type 5, adenovirus type 9, and adenovirus type 12. In a particular embodiment, the adenovirus is adenovirus type 5. GenBank Accession Nos. P68951 and AAW65510.1 provide an example of the amino acid sequence of the precursor protein VII (and mature form) from human adenovirus type 5. In a particular embodiment, the protein VII peptide is acetylated. Nucleic acids encoding the protein VII peptides are also encompassed by the instant invention. Compositions comprising the protein VII peptide and/or nucleic acids encoding the same are also encompassed by the instant invention. The compositions may further comprise at least one other anti-inflammatory agent.

In one embodiment, the isolated protein VII peptide ie is between 66 and 45 amino acids in length inclusive of the N-terminus of protein VII and further comprises a tag sequence. In certain embodiments, the protein VII peptide is acetylated, phosphorylated and/or contains a blocked N-terminus.

The isolated nucleic acid encoding a protein VII peptide is preferably from a human adenovirus and consists of amino acids 1-47, 1-66, or 1-80, operably linked to signal peptide sequence. Such nucleic acids are preferably cloned within an expression vector. The expression vector can be selected from the group consisting of an adenoviral vector, an adeno-associated viral vector, a lentiviral vector, a plasmid vector, a herpes simplex virus vectors, and a vaccinia virus vector. In other embodiments the nucleic acid encoding the protein VII peptide is operably linked to a signal peptide selected from SEQ ID NOS: 27-30.

In accordance with another aspect of the instant invention, methods for reducing, inhibiting, and/or preventing inflammation in a subject are provided. The methods comprise administering protein VII and/or a protein VII peptide (e.g., contained within a composition with a pharmaceutically acceptable carrier) to the subject. The method may further comprise administering at least one other anti-inflammatory agent to the subject.

In accordance with another aspect of the instant invention, methods for treating, inhibiting, and/or preventing an inflammatory disease or disorder in a subject are provided. The method comprises administering a protein VII peptide (e.g., contained within a composition with a pharmaceutically acceptable carrier) peptide to the subject. The method may further comprise administering at least one other anti-inflammatory agent to the subject. In a particular embodiment, the inflammatory disease or disorder is arthritis, sepsis, ARDS, organ failure, ischemia, cancer, infection, colitis, trauma, endotoxemia, sickle cell acute chest syndrome, severe pneumonia, or respiratory tract inflammation.

### BRIEF DESCRIPTION OF THE DRAWINGS

FIGS. 1A-1G show that protein VII is sufficient to alter chromatin and directly binds nucleosomes. Ad5-infected SAECs stained for protein VII with DBP (FIG. 1A), or histone H1 (FIG. 1B), and DAPI. Protein VII-HA induced cells over four days showing HA and DAPI (FIG. 1C). FIG. 1D: SDS-PAGE of histone extract from Ad5-infected cells showing protein V and protein VII. FIG. 1E: Western blot of chromatin fractionation from nuclei of Ad5-infected cells, induced for protein VII-HA, or untreated. FIG. 1F: Protein VII binds to nucleosomes. Protein bands from native gel stained with coomassie (top) were subjected to 2D analysis by SDS-PAGE (bottom). FIG. 1G: Protein VII protects nucleosome complexes from MNase digestion. Bioanalyzer curves represent nucleosomes alone or protein VII-nucleosome complexes. FIG. 1H: Protein VII localizes to cellular chromatin and viral replication centers in U2OS similarly to SAECs in FIG. 1A. FIG. 1I: Protein VII mRNA levels measured by quantitative PCR showing that after 4 days of induction in the A549 cell line, the level of protein VII transcripts is approximately 10% of that measured during infection at 16 hpi. Despite the low relative level, this amount of protein VII is sufficient to cause dramatic changes in the nucleus (error bars=±s.d., n_{biological}=3). FIG. 1J: Inducible cell lines of U2OS and HeLa expressing protein VII-HA show chromatin localization and distortion, similar to A549 cells in FIG. 1C. FIG. 1K: Inducible A549 cell lines expressing viral protein V, the precursor for protein VII (preVII) or cellular protamine PRM1 with C-terminal HA tags. Although all 3 proteins possess a large number of charged residues, none are sufficient to distort cellular chromatin or increase nuclear size as observed with mature protein VII. FIG. 1L: Western blot analysis showing protein VII in histone extracts from infected HeLa cells at 24 hpi. FIG. 1M: Chromatin fractionation of lysates from A549 cells that were uninfected (mock) or infected for 24 hours with Ad5. Viral and cellular proteins were detected by western blotting with various antibodies as indicated. FIG. 1N: Agarose gel analysis of DNA extracted from nuclear fractionation experiments indicating the size of DNA is between 100-200 bp and elutes predominantly in the higher salt fractions. FIG. 1O: Chromatin fractionation of cells induced to express protein VII indicating protein VII present in the highest salt fraction from the first day of induction. FIG. 1P: Recombinant protein VII-His binds DNA. Incubating increasing molar amounts of protein VII with 195 bp DNA results in shifts by native gel electrophoresis indicating protein VII-DNA complex formation. Staining with either ethidium bromide (FIG. 1P) or coomassie (FIG. 1Q) are shown to verify the presence of DNA and protein, respectively. FIG. 1R: ethidium bromide staining shows DNA content of nucleosome shifts from gel in FIG. 1F. FIG. 1S: 195 bp nucleosomes or protein VII-nucleosome complexes were incubated with MNase for the indicated times, the reaction was stopped, DNA extracted and analyzed. As in FIG. 1G, nucleosomes and protein VII-nucleosome complexes are shown. The presence of protein VII pauses digestion at 165 bp, indicating that protein VII is blocking access to the DNA. FIG. 1T: 147 bp nucleosomes or protein VII-nucleosome complexes were incubated with MNase for the indicated times, the reaction was stopped, DNA extracted and analyzed. Graphs show nucleosomes and protein VII-nucleosome complexes. The presence of protein VII completely blocks digestion even after nucleosomes alone have been digested well beyond the core particle. In contrast to what would be expected for linker histones, protein VII protects the core nucleosome particle from digestion. These data indicate that protein VII masks the substrate for MNase through complex formation. This represents a unique mechanism of nucleosome binding and suggests a model for blocking DNA access in cellular chromatin during infection.

FIG. 2A: RP-HPLC analysis of histone extracts. Viral proteins V, VII and preVII are indicated at 24 hpi. FIG. 2B: Primary sequence of protein VII with modified residues identified in infected cells (SEQ ID NO:1). Underlined residues represent moieties that may also be modified in identified peptides. FIG. 2C: Immunofluoresence showing DAPI and protein VII as wild-type or with alanine substitutions at post-translational modification (PTM) sites (APTM), K3A or K3Q. FIG. 2D: Coomassie stained SDS-PAGE analysis of fractions from RP-HPLC in FIG. 2A. The bands in fraction 38-41 min correspond to histone H1. Protein VII and V, as indicated, were verified by mass spectrometry analysis. The slight upward shift of the protein VII bands in the later peak corresponds to the higher abundance of protein preVII, as seen by HPLC in FIG. 2A. FIG. 2E: Western blot analysis of protein VII in HPLC fractions from FIG. 2D. FIG. 2F: Time-course of infection followed by histone extraction and HPLC analysis. Mass spectrometry analysis verified peaks in each sample as indicated. FIG. 2G: Representative mass spectra. Annotated MS/MS spectra of identified peptides of protein VII containing PTMs (acetylated peptides and phosphorylated peptides). The images represent the observed fragment ions collected using MS/MS collision induced dissociation (CID). Lines represent matches between observed and expected fragment ions of the given peptides. FIG. 2H: LC-MS analysis of unmodified and modified chymotryptic peptide AKKRSDQHPVRVRGHY (SEQ ID NO: 3). On the left, nanoLC-MS extracted ion chromatograms of protein VII peptides identified in the histone extracts of adenovirus infected cells (Inf) or viral particles (VP). The top left represents the modified form, while the bottom left the unmodified form. Non-modified forms were detected in both conditions for VII and VP, while the acetylated form was unique for the infected sample only (Inf). On the right, full MS spectrum of the modified (top) and unmodified (bottom) peptide. Circled mass represents the monoisotopic signal of the peptide. FIG. 2I: Summary of post-translational modifications detected on protein VII. Peptides shown were identified during infection at various time points with the mature protein VII in the top row and the precursor, preVII, in the bottom row. The numbers in brackets for preVII indicate the location of the same moiety in mature protein VII. Acetylation sites were detected in approximately 3% of peptides for mature protein VII and 2% of peptides in preVII. Phosphorylation was detected in approximately 1% of peptides for mature protein VII and preVII (SEQ ID NOS: 3-9 are shown). FIGS. 2J-2K: Quantification of histone H3 (FIG. 2J) and H4 (FIG. 2K) PTMs in protein VII-HA induced (+dox) and uninduced (−dox) A549 cells from the analysis of crude histone mixtures (n_{biological}=3). Positions of PTMs are listed along the x-axis. Modification type is indicated as shown. y-axis represents the cumulative extent of PTMs as relative to the total histone H3 or H4, respectively. FIG. 2L: Breakdown of the histone marks (H3K14ac, H3K27me1, H3K36me3, H4K20me1, H4K20me2, and H4K20me3) found significantly different (n_{biological}=3) in terms of relative abundance between the protein VII-HA induced and uninduced states (<5% homoscedastic two-tailed t-test). Error bars represent ±standard deviation.

FIGS. 3A-3K: Protein VII directly binds HMGB1 and is necessary for retention of the alarmin in cellular chromatin. FIG. 3A: Volcano plot for proteomics analysis of one representative biological replicate of high salt fraction. The y-axis represents −log₂ statistical p-value and x-axis represents log₂ protein fold-change between uninduced or protein VII expressing cells (homoscedastic two-tailed t-test, p<0.05 red dots; n_{technical}=3). FIG. 3B: Nuclear fractionation shows HMGB1 and HMGB2 normally elute from nuclei at low salt concentrations but are retained in high salt fractions by protein VII-HA. FIG. 3C: Protein VII interacts with HMGB1 in pull-down of recombinant HMGB1-GST (left, coomassie-stained SDS-PAGE) and immunoprecipitation of HMGB1 (right, western blots). FIG. 3D-3E: Protein VII expression alters localization of HMGB1 (FIG. 3D) and HMGB2 (FIG. 3E). Immunofluorescence shows protein VII-HA co-localized with HMGB1 (FIG. 3D) and HMGB2 (FIG. 3E) in cellular chromatin, DAPI. FIG. 3F: Same as FIG. 3D at 18 hpi with Ad5 DBP. FIG. 3G: Protein VII-GFP relocalizes HMGB1 to chromatin with DAPI. FIG. 3H: FRAP experiment with HMGB1-mGFP. Recovery of FRAP signal in time-course images (left) with quantification and diffusion coefficients (right). FIG. 3I: Schematic showing loxP strategy for deleting protein VII. FIG. 3J: Western blots comparing 293 and 293Cre cells infected with Ad5-flox-VII virus. FIG. 3K: Salt fractionation in nuclei from FIG. 3J. FIG. 3L: Venn diagram showing overlap between three biological replicates of high salt fraction proteins significantly enriched as compared to uninduced cells. FIG. 3M: Proteins found significantly enriched in the protein VII-HA induced state as compared to uninduced (<5% homoscedastic t-test) in all three biological replicates (VII-HA induced: proteins identified only in protein VII-HA induced condition). FIG. 3N-30: Classification of proteins significantly enriched in minimum two out of three biological replicates (protein VII-HA induced vs uninduced) according to process network enrichment and gene ontology (GO) biological process (GeneGo's MetaCore pathways analysis package; false discovery rate <5%); each GO term was ranked using p-value enrichment. FIG. 3P: Western blot of adenovirus infected or doxycycline treated A549 cells showing the relative levels of protein VII expression. HMGB1 levels do not change upon infection or protein VII expression. Tubulin is shown as a loading control. FIG. 3Q: Quantitative PCR analysis of mRNA transcripts of HMGB1 in various cell types as indicated (for A549, n_{biological}=3, for THP-1, n_{biological}=2, error bar=±s.d.). The levels of HMGB1 do not significantly change. FIG. 3R: Immunofluorescence analysis of a time-course of protein VII-HA induction shown with HMGB1 and DAPI in A549 cells. Expression of protein VII-HA results in a change to the HMGB1 distribution upon expression. FIG. 3S: HMGB1 localization changes between 12 and 24 hpi of wild-type adenovirus in A549 cells, and adopts a pattern similar to protein VII as in FIG. 1A. DBP is shown as a marker of infection, DNA is stained with DAPI. FIG. 3T: Same as FIG. 3S showing HMGB2 adopts the same pattern as HMGB1 during Ad5 infection at 24 hpi. FIG. 3U: Multiple cells showing the same pattern of HMGB1 re-localization upon expressing VII-GFP as in FIG. 3G. FIG. 3V: HMGB1 retention in the high salt fraction is conserved across Ad serotypes. Western blot analysis of HMGB1 from salt fractionated A549 cells infected with Ad5, Ad9 or Ad12 as shown.

FIG. 4A: Precision cut lung slices (PCLS) infected with Ad5 or transduced to express protein VII-GFP. Endogenous HMGB1 is redistributed in cells with virus (DBP in top) and VII-GFP (bottom). FIG. 4B: Protein VII-GFP is sufficient to inhibit HMGB1 and HMGB2 release in THP-1 cells. Numbers indicate relative intensities of bands quantified with Image.” FIG. 4C: ELISA-based quantification of HMGB1 in supernatants from FIG. 4B, error bars=±s.d., n_{technical}=4, homoscedastic one-tailed t-test. FIG. 4D: Schematic for investigating protein VII in a mouse lung injury model. FIG. 4E: Expression of protein VII-GFP decreases HMGB1 in mouse BAL fluid as quantified by ELISA, error bars=±s.d., biological replicates: nLPS=4, nGFP+LPS=6, nVII-GFP+LPS=7, homoscedastic one-tailed (p=0.02) or two-tailed (p=0.003) t-test. FIG. 4F: Neutrophils in BAL fluid are significantly fewer in mice expressing protein VII-GFP, error bars=±s.d., biological replicates: nGFP+LPS=6, nVII-GFP+LPS=4, nLPS=5, nGFP=3, nVII-GFP=3 homoscedastic two-tailed t-test. FIGS. 4G-4H: Replication of Ad5-flox-VII virus on 293 or 293-Cre cells. Quantitative PCR analysis of viral genomic DNA over a time-course of infection (FIG. 4G) shows the DBP gene is increasing exponentially in 293 and 293-Cre cells when infected with Ad5-flox-VII virus. In contrast, PCR for the protein VII gene (FIG. 4H) demonstrates deletion in 293-Cre cells (n_{biological}=2, error bar=±s.d.). FIG. 4I: Salt fractionation of 293-Cre cells infected with wild-type Ad5 indicating that the Cre recombinase does not interfere with the ability of protein VII to retain HMGB1 in the high salt chromatin fraction. Protein VII is also necessary for the chromatin retention of HMGB2. FIG. 4J: THP-1 cells transduced to express protein VII-GFP results in chromatin distortion and HMGB1 retention in chromatin. Immunofluorescence of transduced PMA-treated THP-1 cells showing protein VII-GFP, HMGB1 and DNA. FIG. 4K: Transduction to express protein VII-GFP is sufficient to relocalize mouse HMGB1 in mouse embryonic fibroblast (MEF) cells. FIG. 4L: Salt fractionation of mouse embryonic fibroblast cells transduced to express protein VII-GFP. Human Ad5 protein VII is sufficient to retain mouse HMGB1 in the high salt fraction in MEF cells. The control vector expressing GFP alone does not have this effect. FIG. 4M: Sections of mouse lungs transduced to express protein VII-GFP or GFP co-stained for HMGB1. GFP signal shows multiple cell types transduced in both cases. Protein VII-GFP has a more distinct nuclear signal than GFP, which also appears cytoplasmic. Two sections for each condition are shown to indicate transduction efficiency. FIG. 4N: Same as FIG. 4M but co-stained for prosurfactant-C to mark type II pneumocytes. Some cells are positive for both, confirming multiple cell types transduced. FIG. 4O: Zoomed images of individual epithelial cells from mouse lungs showing the characteristic protein VII-GFP pattern colocalizing with DAPI in the nucleus. GFP only is mostly cytoplasmic. FIG. 4P: Schematic summarizing function of protein VII during infection. Newly synthesized protein VII late during infection can be post-translationally modified and binds to HMGB1, sequestering it on the cellular chromatin and preventing its release. Unmodified protein VII is packaged in viral progeny.

FIGS. 5A-5B: Human, but not mouse adenoviral protein VII retains HMGB1 in chromatin. FIG. 5A: Western blot of chromatin fractionation of A549 cells uninduced (control) and induced for expression of protein VII-HA from human adenovirus serotype 5 (Ad5 VII-HA) or protein VII-HA from mouse adenovirus serotype 1 (MAV-1 VII-HA) by doxycycline treatment for 4 days. FIG. 5B: Immunofluorescence of HMGB1 in A549s cells under control conditions or upon expression of Ad5 or MAV-1 protein VII-HA. HMGB1 has a pan-nuclear staining in control cells.

FIGS. 6A-6B: The first 66 amino acids of human adenovirus protein VII have the ability to relocalize HMGB1. FIG. 6A: Schematic of the different chimeras between Ad5 and MAV-1 protein VII indicating the regions of each protein present in the construct, the localization within the cell and the ability to relocalize HMGB1. FIG. 6B: Immunofluorescence of HMGB1 in A549s cells under control conditions or upon expression the different protein VII chimeras.

FIGS. 7A-7C: The first 47 amino acids of human adenovirus protein VII bind HMGB1 in cells. FIG. 7A: Schematic of the different Ad5 protein VII constructs tagged with GFP. FIG. 7B: GFP immunoprecipitation from A549 cells transfected for 24 hours with GFP or the different protein VII-GFP constructs. FIG. 7C: Cellular fractionation of A549 cells transfected for 24 hours with GFP and the different protein VII constructs.

FIGS. 8A-8C: Protein VII relocalized the HMGB1 A box but not the B box or acidic tail. FIG. 8A: Schematic of the different GFP constructs expressing distinct HMGB1 domains. FIG. 8B: Immunofluorescence of the HMGB1-GFP constructs 2 days after transfection of A549s cells under control conditions or upon expression of Ad5 protein VII-HA. Scale bar=10 μm. FIG. 8C: Western blot showing the expression levels of the different HMGB1-GFP constructs.

FIG. 9 is a micrograph showing that protein VII immobilized HMGB1 A box in chromatin similar to that observed with full length protein. Immuno-fluorescence time course showing the fluorescence recovery after photo-bleaching of full-length HMGB1 or just the HMGB1 A box.

FIG. 10 shows an in vitro GST pull down assay that demonstrates that protein VII binds to the HMGB1 acidic tail in vitro.

FIG. 11A is schematic diagram of the interaction of the first 47 amino acids of protein VII which bind both the HMGB1 A box as well as the acidic tail in a 3D structure, thereby covering the immune receptor binding sites present in HMGB1.

FIG. 11B provides exemplary signal sequences for use as fusions with protein VII peptides that are delivered in viral vectors. SEQ ID NOS:27-30 are shown.

### DETAILED DESCRIPTION OF THE INVENTION

Viral proteins mimic host protein structure and function to redirect cellular processes and subvert innate defenses (Elde et al. (2009) Nat. Rev. Microbiol. 7:787-797). Small basic proteins compact and regulate both viral and cellular DNA genomes. Nucleosomes are the repeating units of cellular chromatin and play an important role in innate immune responses (Smale et al. (2014) Annu. Rev. Immunol., 32:489-511). Viral encoded core basic proteins compact viral genomes but their impact on host chromatin structure and function was not known. Adenoviruses encode a highly basic protein called protein VII that resembles cellular histones (Lischwe et al. (1977) Nature 267:552-554). Although protein VII binds viral DNA and is incorporated with viral genomes into virus particles (Chatterjee et al. (1986) EMBO J., 5:1633-1644; Vayda et al. (1983) Nuc. Acids Res., 11:441-460), it was unknown whether protein VII impacts cellular chromatin.

Here, it was determined that protein VII alters cellular chromatin and, thus, impacts antiviral responses during adenovirus infection. It was found that protein VII forms complexes with nucleosomes and limits DNA accessibility. Post-translational modifications on protein VII that are responsible for chromatin localization have been identified. Furthermore, proteomic analysis demonstrated that protein VII is sufficient to alter protein composition of host chromatin. Protein VII is necessary and sufficient for retention in chromatin of members of the high-mobility group protein B family (HMGB1, HMGB2, and HMGB3). HMGB1 is actively released in response to inflammatory stimuli and functions as a danger signal to activate immune responses (Kang et al. (2014) Mol. Aspects Med., 40:1-116; Lotze et al. (2005) Nat. Rev. Immunol., 5:331-342). It is also shown that protein VII can directly bind HMGB1 in vitro and that protein VII expression in mouse lungs is sufficient to decrease inflammation-induced HMGB1 content and neutrophil recruitment in the bronchoalveolar lavage fluid. Together the in vitro and in vivo results show that protein VII sequesters and/or inhibits HMGB1 and can prevent its release. Protein VII can also sequester and/or inhibit HMGB2 and/or HMGB3. This shows a viral strategy in which nucleosome binding is exploited to control extracellular immune signaling.

As stated above, it has been shown herein that adenovirus protein VII binds to HMGB1 in the nucleus and prevents it from being released into the extracellular space. This interaction serves to attenuate the resulting inflammatory response. It has been shown that protein VII, delivered in the absence of a viral infection, is able to bind HMGB1 both in vitro and in vivo and prevent HMGB1 release in to the extracellular compartment. Protein VII binds to HMGB1 through a domain that is at most about 80 amino acids, at most about 70 amino acids, at most about 66 amino acids, at most about 60 amino acids, at most about 50 amino acids, or at most about 40 amino acids. In a particularly preferred embodiment, the protein VII is a fragment of 47 amino acids. This indicates that smaller peptide molecules/fragments can serve a similar function. In addition to the above, protein VII was expressed in the lung using a recombinant adenovirus (rAd). The mouse was then exposed to inhaled LPS to lead to inflammation in the lung. In mice expressing protein VII, the release of HMGB1 and the resulting inflammation was attenuated compared with mice expressing a control protein (GFP).

Accordingly, protein VII peptides can be used to block circulating HMGB1, HMGB2, and/or HMGB3 binding to receptors to dampen amplification of HMGB-mediated inflammation. Methods of screening for additional protein VII peptide fragments which modulate (e.g., inhibit) HMGB (e.g., HMGB1) are encompassed by the instant invention. For example, the methods used in the Example may be used (e.g., HMGB1 localization, HMGB1 co-immunoprecipitation, etc.). Peptides (e.g., minimal peptides) that can bind HMGB1 and block its binding to TLR and RAGE receptors may be identified using reporter cells. For example, peptides can be incubated with recombinant HMGB1 and then added to the supernatants of cells (e.g., reporter 293 cells) that secrete a reporter (e.g., secreted alkaline phosphatase) downstream which can be measured (e.g., using a commercially available detection kit). The pharmacokinetics of a peptide may also be tested in vivo where toxicity and half life can be measured in, for example, mice (see, e.g., the Example). The mice can be exposed to LPS inhalation and the inflammatory response in the lung fluid can be measured to determine the efficacy of the peptide. The protein VII peptides described herein should also be effective to decrease inflammation post-injury. Since HMGB1 is a mediator of inflammation in many diseases, the peptide should also have efficacy in other inflammatory disorders such as sepsis. The peptides may be post-translationally modified as protein VII is in vivo. While mature protein VII and peptides thereof are described throughout the application, the instant invention also encompasses precursor protein VII and peptides/fragments thereof (e.g., optionally comprising the same modifications as described herein for the mature protein VII peptides).

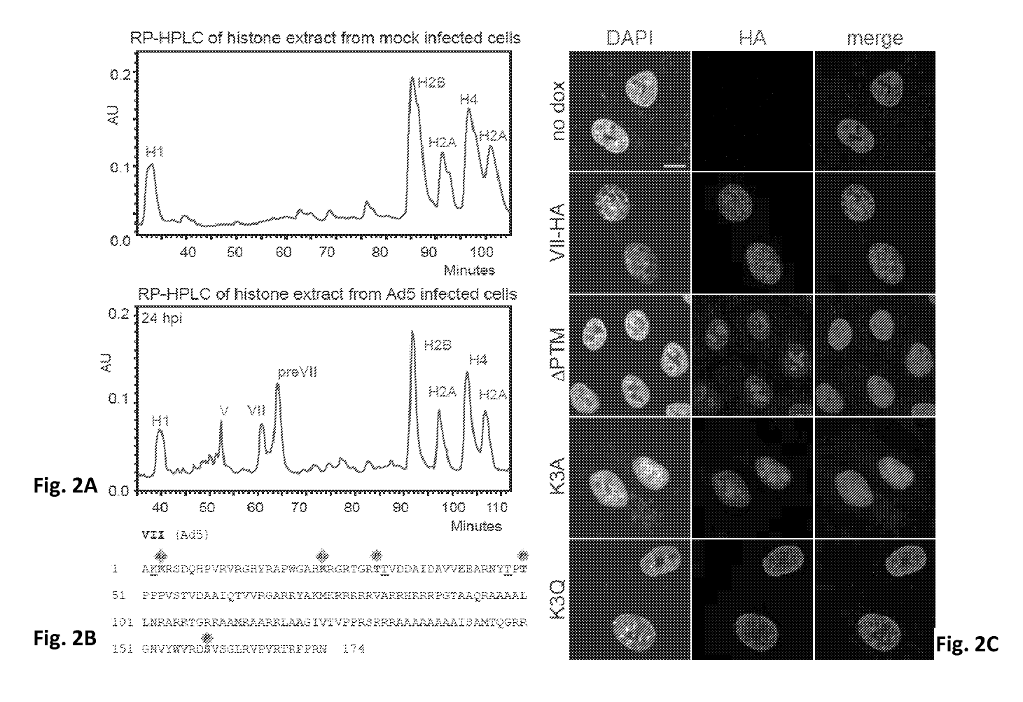

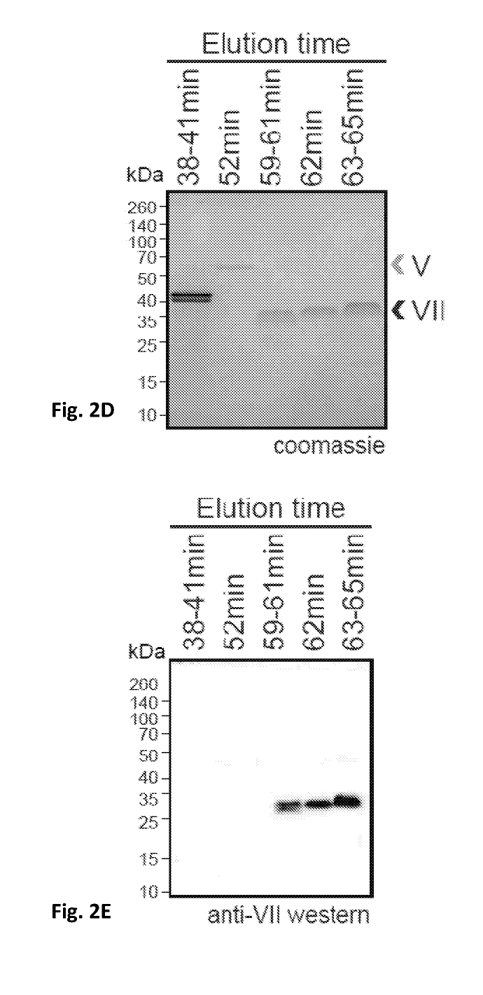

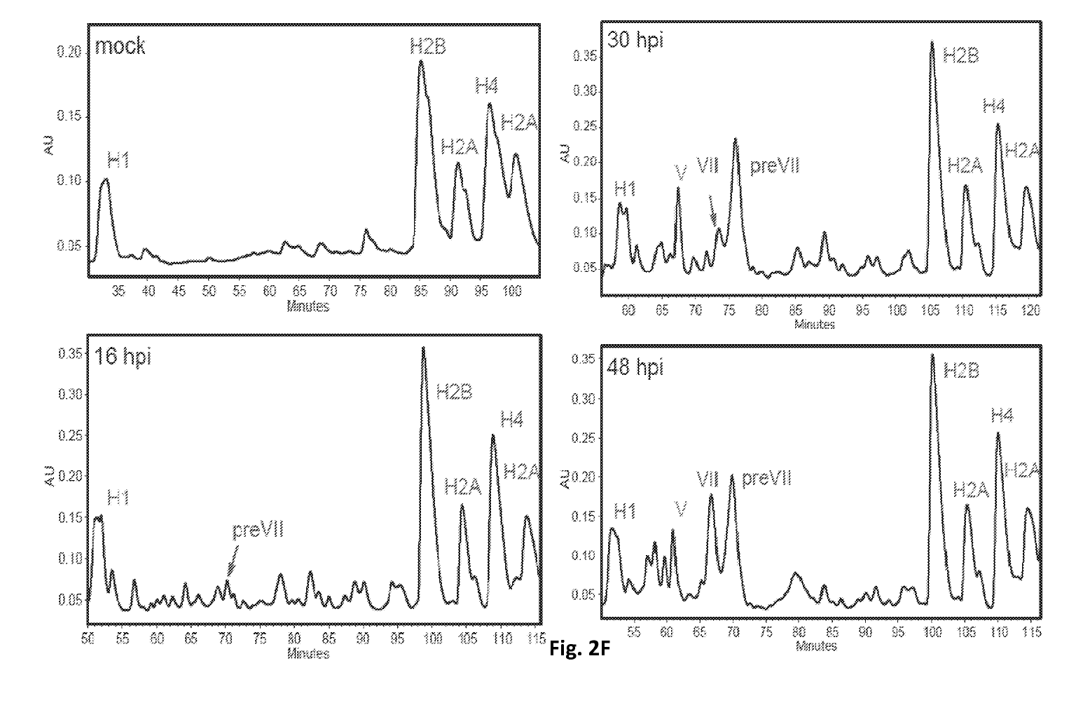

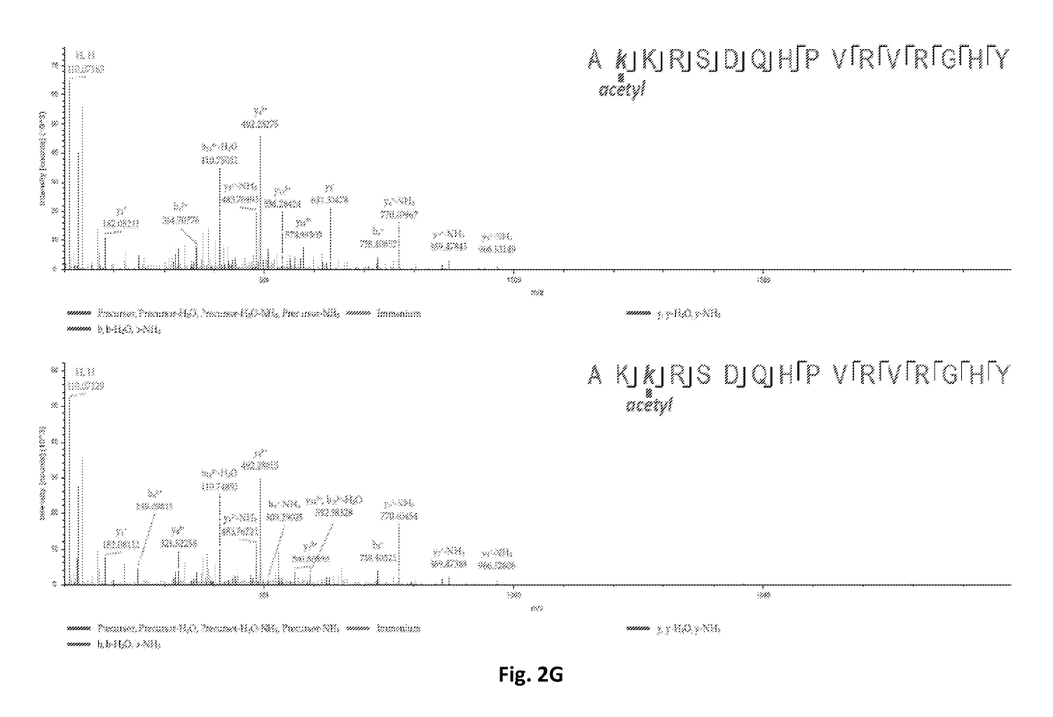

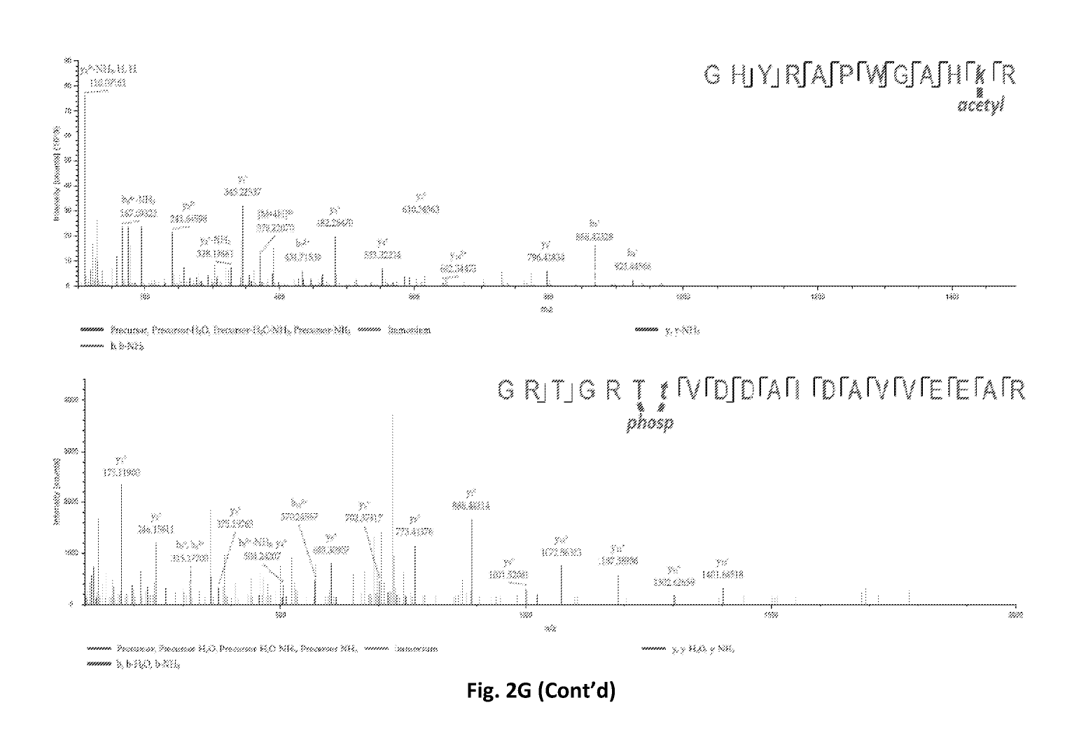

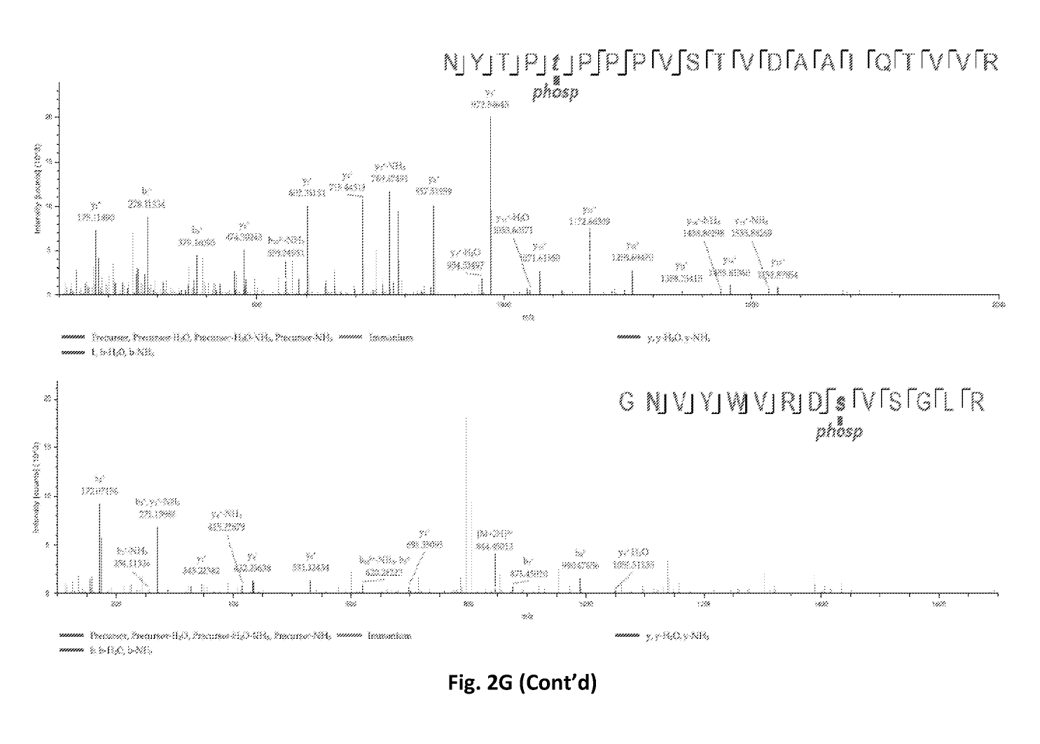

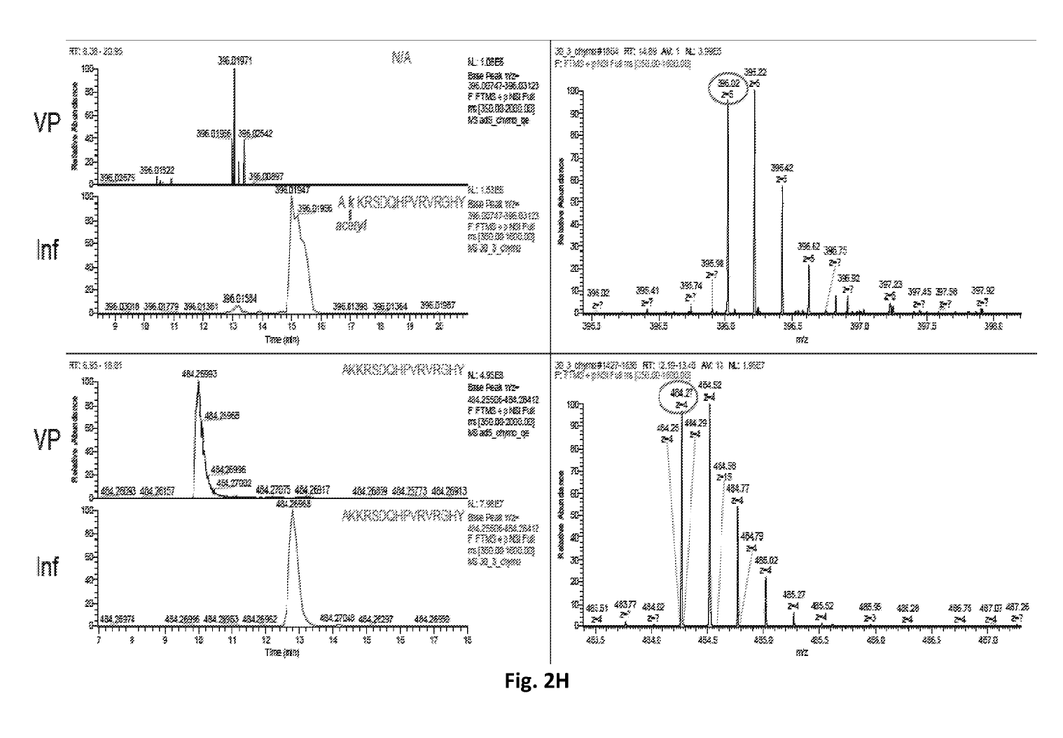

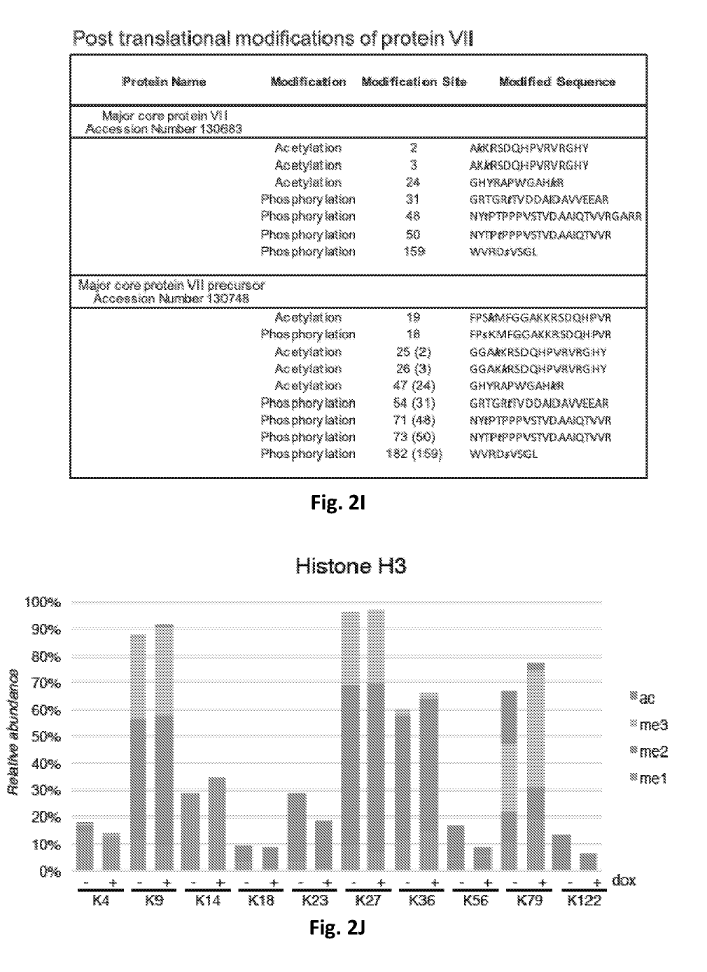

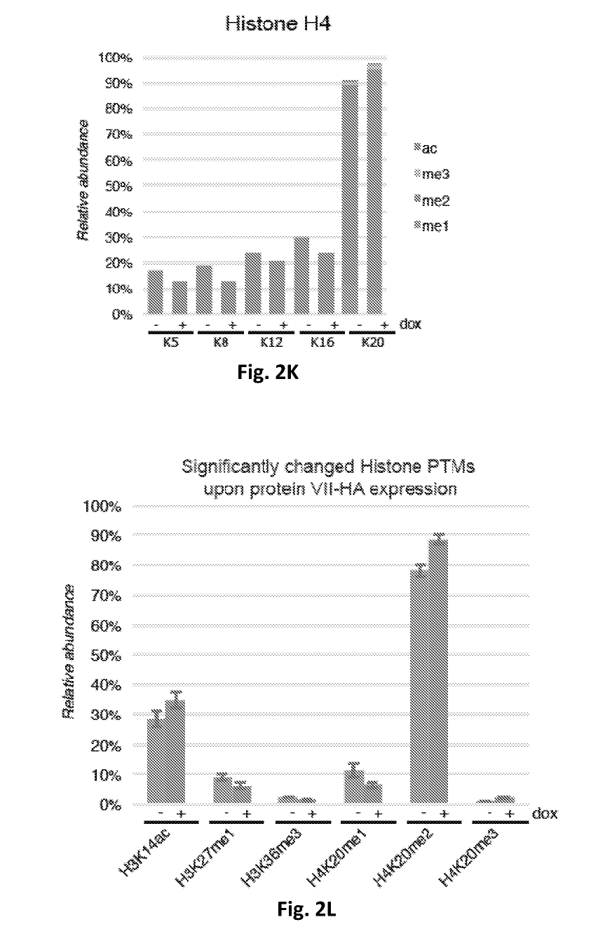

In accordance with an aspect of the instant invention, protein VII peptides are provided. The protein VII can be from any adenovirus serotype. In a particular embodiment, the protein VII is from adenovirus type 5. The full length amino acid sequence of protein VII is provided in FIG. 2B. In a particular embodiment, the protein VII peptides of the instant invention have at least 50%, 75%, 80%, 85%, 90%, 95%, 97%, 99%, or 100% homology or identity with the sequence provided in FIG. 2B. For example, the protein VII peptides of the instant invention may have insertions, deletions, and/or substitutions or may be truncated compared to the amino acid sequence of FIG. 2B.

Protein VII peptides may be from about 10 to about 100 amino acids, about 10 to about 75 amino acids, about 10 to about 70 amino acids, about 10 to about 66 amino acids, about 10 to about 60 amino acids, about 10 to about 50 amino acids, about 10 to about 45 amino, about 10 to about 40 amino acids, about 10 to about 35 amino acids, about 10 to about 30 amino acids, or about 10 to about 25 amino acids in length. In a particular embodiment, the protein VII peptide is less than about 100 amino acids, less than about 75 amino acids, less than about 70 amino acids, less than about 66 amino acids, less than about 60 amino acids, less than about 50 amino acids, less than about 40 amino acids, less than about 35 amino acids, less than about 30 amino acids, or less than about 25 amino acids in length. In a particular embodiment, the protein VII peptide is more than about 10 amino acids, more than about 15 amino acids, more than about 20 amino acids, or more than about 25 amino acids in length.

In a particular embodiment, the protein VII peptide comprises the N-terminal half of protein VII. In a particular embodiment, the protein VII peptide is a fragment of the N-terminal half (e.g., amino acids 1-47 of the amino acid sequence provided in FIG. 2B; AKKRSDQHPVRVRGHYRAPWGAHKRGRTGRTTVDD AIDAVVEEARNY; SEQ ID NO: 10). This sequence can include, 1, 2, 3, 4, 5, 6, 7, 8, 9, 10, 11, 12, 20, 30, 40, or 50 additional protein VII amino acids. In a particular embodiment, the protein VII peptide comprises at least the 10 N-terminal amino acids of protein VII (e.g., AKKRSDQHPV; SEQ ID NO: 11), at least the 15 N-terminal amino acids of protein VII (e.g., AKKRSDQHPVRVRGH; SEQ ID NO: 12), at least the 20 N-terminal amino acids of protein VII (e.g., AKKRSDQHP VRVRGHYRAPW; SEQ ID NO: 13), at least the 25 N-terminal amino acids of protein VII (e.g., AKKRSDQHPVRVRGHYRAPWGAHKR; SEQ ID NO: 14), at least the 30 N-terminal amino acids of protein VII, at least the 35 N-terminal amino acids of protein VII, at least the 40 N-terminal amino acids of protein VII, at least the 45 N-terminal amino acids of protein VII, at least the 50 N-terminal amino acids of protein VII, at least the 55 N-terminal amino acids of protein VII, at least the 60 N-terminal amino acids of protein VII, at least the 66 N-terminal amino acids of protein VII, or at least the 70 N-terminal amino acids of protein VII. In a particular embodiment, the protein VII peptide comprises amino acids 23-66 of the amino acid sequence provided in FIG. 2B (e.g., HKRGRTGRTTVDDAID AVVEEARNYTPTPPPVSTVDAAIQTVVR; SEQ ID NO: SEQ ID NO: 15). In particular embodiment, the protein VII peptide comprising amino acids 23-66 is extended 1, 2, 3, 4, 5, 6, or 7 in the N-terminal and/or C-terminal direction (e.g., along the amino acid sequence provided in FIG. 2B). In a particular embodiment, the protein VII peptides of the instant invention comprises an amino acid sequence having at least 75%, 80%, 85%, 90%, 95%, 97%, 99%, or 100% homology or identity with amino acids 23-66 of the amino acid sequence provided in FIG. 2B.

The protein VII peptides of the instant invention may have the same post-translational modifications as protein VII (see, e.g., FIG. 2B). For example, the protein VII peptides may have the same acetylation and phosphorylation patterns as full-length protein VII. In a particular embodiment, the amino acid of the protein VII peptide corresponding to K2, K3, and/or K24 (e.g., of the amino acid sequence of FIG. 2B) is acetylated. In a particular embodiment, the amino acid of the protein VII peptide corresponding to K3 (e.g., of the amino acid sequence of FIG. 2B) is acetylated. In a particular embodiment, the amino acid of the protein VII peptide corresponding to K24 (e.g., of the amino acid sequence of FIG. 2B) is acetylated. In a particular embodiment, the amino acid of the protein VII peptide corresponding to K2 (e.g., of the amino acid sequence of FIG. 2B) is acetylated. In a particular embodiment, the amino acid of the protein VII peptide corresponding to T31, T32, T48, and/or T50 (e.g., of the amino acid sequence of FIG. 2B), particularly T31, T48, and/or T50, is phosphorylated.

As stated hereinabove, the peptides of the instant invention may contain substitutions for the amino acids of the provided sequence. These substitutions may be similar to the amino acid (i.e., a conservative change) present in the provided sequence (e.g., an acidic amino acid in place of another acidic amino acid, a basic amino acid in place of a basic amino acid, a large hydrophobic amino acid in place of a large hydrophobic, etc.). The substitutions may also comprise amino acid analogs, non-natural amino acids, derivative of standard amino acids (e.g., fluorinated residues or nonstandard amino acids, including beta-amino acids), and/or mimetics.

In a particularly preferred embodiment, the protein VII peptide sequences are modified to eliminate any trypsin or chymotrypin cleavage sites by modifying the sequence such that they are no longer cleavable by these enzymes. This can be achieved by substituting the amino acids in the cleavage site with amino acids that are not cleaved by trypsin or chymotrypsin but result in a modified protein VII peptide which retains the HMGB1 binding of the 47 mer peptide described herein.

The peptides of the instant invention may have capping, protecting and/or stabilizing moieties at the C-terminus and/or N-terminus. Such moieties are well known in the art and include, without limitation, amidation and acetylation. The peptide template may also be lipidated or glycosylated at any amino acid (i.e., a glycopeptide). The peptides may be PEGylated to improve druggability. The number of the PEG units (NH₂(CH₂CH₂O)CH₂CH₂CO) may vary, for example, from 1 to about 50. The peptides of the instant invention may also comprise at least one D-amino acid. The peptide may comprise only D-amino acids.

In a particular embodiment, the peptide may also be circulated or cyclized head to tail or locally involving less than the entirety of amino acid residues. Methods of cyclizing peptides are known in the art.

Due to our finding that protein VII can disrupt nuclear structure, in a preferred embodiment, the biologically active protein VII peptide is directed to the extracellular space to avoid any off target infects within the cell via inclusion of a signal peptide. Thus when a vector based expression systems is employed to introduce protein VII 1-47 a secretion signal is included to ensure the release of the peptide to the extracellular space, where it can block HMGB1 binding to receptors and decrease inflammation. Exemplary signal sequences are provided in FIG. 11B. However, a number of such sequences are available to the skilled artisan and are also within the scope of the invention. If the peptides are to be delivered in the absence of a viral vector, (e.g., infused, inhaled or injected into the subject) a tag sequence is fused with the peptide. Suitable tag sequences include without limitation, FLAG, HIS, HA, Biotin, and GFP.

The peptides of the present invention may be prepared in a variety of ways, according to known methods. The peptides of the instant invention may be made by chemical peptide synthesis (e.g., solid phase synthesis). The availability of nucleic acid molecules encoding the peptide also enables production of the protein using in vitro expression methods and cell-free expression systems known in the art. In vitro transcription and translation systems are commercially available. The peptides may also be produced by expression in a suitable prokaryotic or eukaryotic system. For example, part or all of a DNA molecule encoding for the peptide may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences. The peptides produced by gene expression in a recombinant prokaryotic or eukaryotic system may be purified according to methods known in the art. The peptides of the invention, prepared by the aforementioned methods, may be analyzed according to standard procedures. For example, such peptides may be subjected to amino acid sequence analysis, according to known methods.

Compositions comprising at least one protein VII peptide are also encompassed by the instant invention. The present invention also encompasses nucleic acids encoding the peptides of the invention as well as compositions comprising at least one nucleic acid encoding a protein VII peptide of the instant invention. Nucleic acids of the present invention may be maintained in any convenient vector (e.g., viral vector (e.g., AAV)), particularly an expression vector. Different promoters may be utilized to drive expression of the nucleic acid sequences based on the cell in which it is to be expressed. As mentioned above, Protein VII peptides expressed in viral vectors will be fused to a signal peptide sequence to ensure transfer outside of the cell producing the peptide. Antibiotic resistance markers may also included in these vectors to enable selection of transformed cells. Protein VII peptide encoding nucleic acid molecules of the invention include cDNA, genomic DNA, RNA, and fragments thereof which may be single- or double-stranded. Thus, this invention provides oligonucleotides having sequences capable of hybridizing with at least one sequence of a nucleic acid molecule of the present invention.

The compositions of the instant invention may comprise at least one carrier, particularly at least one pharmaceutically acceptable carrier. The compositions of the instant invention may also comprise at least one other anti-inflammatory agent. Alternatively, the other anti-inflammatory agent may be contained within a separate composition(s) with at least one carrier, particularly a pharmaceutically acceptable carrier. The composition(s) comprising at least one protein VII peptide (and/or encoding nucleic acid molecule) and the composition(s) comprising at least one other anti-inflammatory agent may be contained within a kit. Such composition(s) may be administered, in a therapeutically effective amount, to a patient in need thereof for the treatment of an inflammatory disease or disorder.

In accordance with another aspect of the instant invention, methods for reducing, inhibiting, and/or preventing inflammation in a subject are provided. The method comprises administering to a subject (e.g., prior to or during the inflammation) a protein VII peptide (and/or encoding nucleic acid molecule). The protein VII peptide may be administered in a composition as described herein. In a particular embodiment, the inflammation is associated with HMGB1, HMGB2, and/or HMGB3. In a particular embodiment, the inflammation is associated with HMGB1. In a particular embodiment, the inflammation is associated with increased HMGB1 activity and/or increased HMGB1 presence in the blood. The inflammation may be associated with an inflammatory disease or disorder. In a particular embodiment, the subject is monitored at least once for reduction in symptoms associated with the inflammatory disease or disorder after administration of the compositions of the instant invention to monitor the treatment, inhibition, and/or prevention of the inflammatory disease or disorder.

In a particular embodiment, the methods for reducing, inhibiting, and/or preventing inflammation may further comprise administering at least one anti-inflammatory agent. As used herein, an “anti-inflammatory agent” refers to compounds for the treatment of an inflammatory disease or the symptoms associated therewith. The protein VII peptides of the instant invention and the other anti-inflammatory agent(s) may be administered together in a single composition or may be administered in separate compositions. Additionally, the protein VII peptides of the instant invention and the other anti-inflammatory agent(s) may be administered at the same time or on different schedules.

As used herein, an “inflammatory disease or disorder” refers to a disease or disorder caused by or resulting from or resulting in inflammation. The term “inflammatory disease or disorder” may also refer to a dysregulated inflammatory reaction that causes an exaggerated response by macrophages, granulocytes, and/or T-lymphocytes leading to abnormal tissue damage and cell death. In a particular embodiment, the inflammatory disease or disorder is associated with HMGB1. An “inflammatory disease or disorder” can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), arthritis, rheumatoid arthritis, tendonitis, bursitis, psoriasis, cystic fibrosis, arthrosteitis, Sjogren's Syndrome, progressive systemic sclerosis (scleroderma), ankylosing spondylitis, polymyositis, dermatomyositis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, Crohn's Disease, colitis (e.g., ulcerative colitis), pernicious anemia, inflammatory dermatoses, usual interstitial pneumonitis (UIP), asbestosis, silicosis, bronchiectasis, berylliosis, talcosis, pneumoconiosis, sarcoidosis, interstitial pneumonia, alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, acute respiratory distress syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (e.g., ischemic injury), allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, osteomylitis, optic neuritis, sepsis, organ failure (e.g., kidney failure), cancer, infection (e.g., microbial infection (e.g., bacteria and/or virus)), trauma (e.g., wounds, injuries, etc.), endotoxemia, sickle cell acute chest syndrome, pneumonia (e.g., severe pneumonia), and respiratory tract (e.g., lung) inflammation. In a particular embodiment, the inflammatory disease or disorder is selected from the group consisting of arthritis, sepsis, ARDS, organ failure (e.g., kidney failure), ischemia, cancer, infection (e.g., microbial infection (e.g., bacteria and/or virus)), colitis, trauma (e.g., wounds, injuries, etc.), endotoxemia, sickle cell acute chest syndrome, pneumonia (e.g., severe pneumonia), and respiratory tract (e.g., lung) inflammation.

In a particular embodiment, the methods for treating, inhibiting, and/or preventing an inflammatory disease or disorder may further comprise administering at least one anti-inflammatory agent. As used herein, an “anti-inflammatory agent” refers to compounds for the treatment of an inflammatory disease or the symptoms associated therewith. The protein VII peptides of the instant invention and the other anti-inflammatory agent(s) may be administered together in a single composition or may be administered in separate compositions. Additionally, the protein VII peptides of the instant invention and the other anti-inflammatory agent(s) may be administered at the same time or on different schedules.

Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, dapsone, infliximab, golimumab, gevokizumab, canakinumab, certolizumab, clenoliximab, efalizumab, eldalumab, etrolizumab, fezakinumab, fletikumab, fontolizumab, tocilizumab, siltuximab, clazakizumab, olokizumab, sarilumab, sirukumab, rituximab, obinutuzumab, ofatumumab, anifrolumab, elsilimomab, alemtuzumab, abrilumab, secukinumab, ixekizumab, brodalumab, gesulkumab, lavrilimumab, lenzilumab, natalizumab, nerelimomab, ocrelizumab, odulimomab, olokizumab, ozanezumab, ozoralizumab, pateclizumab, perakizumab, priliximab, placulumab, rontalizumab, rovelizumab, ruplizumab, sarilumab, sifalimumab, tildrakizumab, toralizumab, ustekinumab, vatelizumab, vedolizumab, visilizumab, zanolimumab, zolimomab, adalimumab, and afelimomab. In a particular embodiment, the anti-inflammatory agent is an antibody based drug (e.g., a monoclonal antibody).

The therapeutic agents of the instant invention (e.g., protein VII peptides or derivatives or mimetics thereof) will generally be administered to a patient (i.e., human or animal subject) in a composition with a pharmaceutically acceptable carrier. For example, therapeutic agents may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of therapeutic agents in the chosen medium may be varied and the medium may be chosen based on the desired route of administration of the pharmaceutical preparation. Except insofar as any conventional media or agent is incompatible with the therapeutic agents, its use in the pharmaceutical preparation is contemplated.

In yet another embodiment, the pharmaceutical compositions of the present invention can be delivered in a controlled release system, such as using an intravenous infusion, an implantable osmotic pump (e.g., a subcutaneous pump), a transdermal patch, liposomes, or other modes of administration. In another embodiment, polymeric materials may be employed. In yet another embodiment, a controlled release system can be placed in proximity of the target tissues of the animal, thus requiring only a fraction of the systemic dose. In particular, a controlled release device can be introduced into an animal in proximity to the desired site.

In another aspect, particularly for the treatment of an inflammatory lung disease, such as pulmonary fibrosis, COPD or ARDS, the peptide may be delivered to the lung in a aerosolized form. A pharmaceutical composition comprising the peptide and optionally, an inflammatory agent, or a viral vector encoding the peptide, can be administered as an aerosol formulation that contains the peptide in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages as drops or sprays. Nasal solutions are generally prepared to be similar to nasal secretions and are generally isotonic and slightly buffered to maintain a pH of about 5.5 to about 6.5, although pH values outside of this range can also be used. Antimicrobial agents or preservatives can also be included in the formulation.

An aerosol formulation for use in inhalations and inhalants is designed so that the peptides are carried into the respiratory tree of the patient. See (WO 01/82868; WO 01/82873; WO 01/82980; WO 02/05730; WO 02/05785. Inhalation solutions can be administered, for example, by a nebulizer. Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the peptides. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The peptides can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as protein VII peptide fragments (e.g., Protein VII 1-47 mer fused to a tag sequence such as FLAG) in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the oligos and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, the oligos. The surfactant can be nonionic, anionic or cationic. One example of an emulsion can include, for example, ethanol, surfactant, water and propellant. Another example of an emulsion can include, for example, vegetable oil, glyceryl monostearate and propane.

Selection of a suitable pharmaceutical preparation will also depend upon the mode of administration chosen. For example, the therapeutic agents may be administered by direct injection into an area proximal to the inflammation or may be delivered systemically or may be inhaled as describe above. When delivered by direct injection, a pharmaceutical preparation comprises the therapeutic agents dispersed in a medium that is compatible with the site of injection. The therapeutic agents may be administered by any method such as intravenous injection into the blood stream, oral administration, inhalation, or by subcutaneous, intramuscular or intraperitoneal injection. Pharmaceutical preparations for injection are known in the art. If injection is selected as a method for administering the therapeutic agents, steps should be taken to ensure that sufficient amounts of the molecules reach their target cells to exert a biological effect.

Pharmaceutical compositions containing the therapeutic agents of the instant invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, direct injection, and intraperitoneal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

The pharmaceutical preparation comprising the active ingredient may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

A “therapeutically effective amount” of a compound or a pharmaceutical composition refers to an amount effective to prevent, inhibit, treat, or lessen the symptoms of a particular disorder or disease.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the terms “host,” “subject,” and “patient” refer to any animal, including mammals such as humans.

The term “isolated protein” or “isolated peptide” refers to a protein/peptide that has been sufficiently separated from other proteins/peptides so as to exist in a “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

A “signal peptide” (sometimes referred to as signal sequence, targeting signal, localization signal, localization sequence, transit peptide, leader sequence or leader peptide) is a short peptide (usually 16-30 amino acids long) present at the N-terminus of the majority of newly synthesized proteins that are destined towards the secretory pathway. These proteins include those that reside either inside certain organelles (the endoplasmic reticulum, golgi or endosomes), secreted from the cell, or inserted into most cellular membranes. N-terminal signal sequences mediate targeting of nascent secretory and membrane proteins to the endoplasmic reticulum (ER) in a signal recognition particle (SRP)-dependent manner. Signal sequences have a tripartite structure, consisting of a hydrophobic core region (h-region) flanked by an n- and c-region. The latter contains the signal peptidase (SPase) consensus cleavage site. Usually, signal sequences are cleaved off co-translationally so that signal peptides and mature proteins are generated. Signal sequences are extremely variable in length and amino acid composition. This variability suggests that ER targeting and the steps beyond like protein insertion and SPase cleavage are affected by the signal sequence. Exemplary signal peptides include, without limitation, those provided in FIG. 11B.

The peptides of the invention may also comprise a sequence “tag” to facilitate detection and purification of the peptide. Suitable tags include without limitation, FLAG, Biotin, HA, GFP, and HIS.

### II. Preparation of Variant Protein VII Encoding Nucleic Acid Molecules and Polypeptides

### A. Nucleic Acid Molecules

Nucleic acid molecules encoding variants of protein VII of the invention may be prepared by using recombinant DNA technology methods. The availability of nucleotide sequence information enables preparation of isolated nucleic acid molecules of the invention by a variety of means. Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Alternatively, the nucleic acids may be maintained in vector suitable for expression in mammalian cells. In cases where post-translational modification affects function, it is preferable to express the molecule in mammalian cells.

### B. Proteins

A variant protein VII polypeptide of the present invention may be prepared in a variety of ways, according to known methods. The protein may be purified from appropriate sources, e.g., transformed bacterial or animal cultured cells or tissues which express protein VII, by immunoaffinity purification.

Larger quantities of protein VII peptide may be produced by expression in a suitable prokaryotic or eukaryotic expression system. For example, part or all of a DNA molecule encoding protein VII for example, may be inserted into a plasmid vector adapted for expression in a bacterial cell, such as E. coli or a mammalian cell such as CHO or Hela cells. Alternatively, in a preferred embodiment, tagged fusion proteins comprising protein VII can be generated as described further hereinbelow. Such protein VII-tagged fusion proteins are encoded by part or all of a DNA molecule, ligated in the correct codon reading frame to a nucleotide sequence encoding a portion or all of a desired polypeptide tag which is inserted into a plasmid vector adapted for expression in a bacterial cell, such as E. coli or a eukaryotic cell, such as, but not limited to, yeast and mammalian cells. Vectors such as those described above comprise the regulatory elements necessary for expression of the DNA in the host cell positioned in such a manner as to permit expression of the DNA in the host cell. Such regulatory elements required for expression include, but are not limited to, promoter sequences, transcription initiation sequences, and enhancer sequences.

Protein VII biologically active fragments, mimetics, or derivatives thereof produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope, GST or the hemagglutinin epitope. Such methods are commonly used by skilled practitioners.

Protein VII proteins, prepared by the aforementioned methods, may be analyzed according to standard procedures. For example, such proteins may be subjected to amino acid sequence analysis, according to known methods.

Accordingly, the present invention also encompasses a method of making a polypeptide (as disclosed), the method including expression from nucleic acid encoding the polypeptide (generally nucleic acid). This may conveniently be achieved by culturing a host cell, containing such a vector, under appropriate conditions which cause or allow production of the polypeptide. Polypeptides may also be produced in in vitro systems, such as in reticulocyte lysates.

The Protein VII peptides can be from any adenovirus, however human adenoviruses are particularly preferred. There are 57 human adenovirus serotypes (HAdV-1 to 57) in seven species (Human adenovirus A to G). The species and serotype numbers are as follows: A: 12, 18, 31; B: 3, 7, 11, 14, 16, 21, 34, 35, 50, 55; C: 1, 2, 5, 6, 57; D: 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56; E: 4; F: 40, 41 and G: 52. Notably, different types/serotypes are associated with different conditions. These include respiratory disease (mainly species HAdV-B and C), conjunctivitis (HAdV-B and D), gastroenteritis (HAdV-F types 40, 41, HAdV-G type 52), and obesity or adipogenesis (HAdV-A type 31, HAdV-C type 5, HAdV-D types 9, 36, 37). Protein VII peptides of at least 80, at least 70, at least 60, at least 50, at least 40, at least 30, at least 20 amino acids from these serotypes are also within the scope of the invention. Preferably, these sequences include the N-terminus and are optionally modified as described herein (e.g., include tags, signal peptides, amino acid substitutions, modifications, etc.). In one aspect, the Protein VII peptides are modified. In certain embodiments, Protein VII peptides are obtained from serotypes associated with inflammation in certain target tissues. For example, serotypes which infect the gut can provide protein VII peptides which are more effective for treatment of disorders associated with gut inflammation, serotypes which infect the respiratory system can provide protein VII peptides which are more effective for treatment of disorders associated with lung inflammation, serotypes which infect the eye can provide protein VII peptides which are more effective for treatment of disorders associated with eye inflammation, etc. Thus, protein VII proteins according to the invention can be chosen based on the type of inflammatory disorder to be treated.

III. Uses of Protein VII Peptides and Protein VII Peptide—Encoding Nucleic Acids

Protein VII nucleic acids encoding biologically active polypeptide fragments having altered HMGB1 binding activities may be used according to this invention, for example, as therapeutic and/or prophylactic agents (protein or nucleic acid) which modulate inflammation in a subject in need thereof. The present inventors have discovered that biologically active fragments of Protein VII directly interact with the HMGB1 highly acidic C-terminus and able to bind both the HGM1A box as well as the acidic tail.

A. Protein VII Polypeptide Fragments

In a preferred embodiment of the present invention, protein VII peptides comprising a tag sequence may be administered to a patient via infusion in a biologically compatible carrier, preferably via intravenous injection. They may also be administered in aerosolized form. The protein VII polypeptides, fragments, mimetics and derivatives of the invention may optionally be encapsulated into liposomes or mixed with other phospholipids or micelles to increase stability of the molecule. Protein VII peptides may be administered alone or in combination with other agents known to modulate inflammation as described herein. An appropriate composition in which to deliver protein VII polypeptides may be determined by a medical practitioner upon consideration of a variety of physiological variables, including, but not limited to, the patient's condition and level of inflammatory disease. A variety of compositions well suited for different applications and routes of administration are well known in the art and are described hereinbelow.

The preparation containing the purified protein VII biologically active fragment or mimetic contains a physiologically acceptable matrix and is preferably formulated as a pharmaceutical preparation. The preparation can be formulated using substantially known prior art methods, it can be mixed with a buffer containing salts, such as NaCl, CaCl₂, and amino acids, such as glycine and/or lysine, and in a pH range from 6 to 8. Until needed, the purified preparation containing protein VII or fragment thereof can be stored in the form of a finished solution or in lyophilized or deep-frozen form. Preferably the preparation is stored in lyophilized form and is dissolved into a visually clear solution using an appropriate reconstitution solution.

Alternatively, the preparation according to the present invention can also be made available as a liquid preparation or as a liquid that is deep-frozen.

The preparation according to the present invention is especially stable, i.e., it can be allowed to stand in dissolved form for a prolonged time prior to application.

The preparation according to the present invention can be made available as a pharmaceutical preparation with protein VII activity in the form of a one-component preparation or in combination with other anti-inflammatory agents in the form of a multi-component preparation.

Prior to processing the purified protein or protein fragment or mimetic into a pharmaceutical preparation, the purified protein is subjected to the conventional quality controls and fashioned into a therapeutic form of presentation. In particular, during the recombinant manufacture, the purified preparation is tested for the absence of cellular nucleic acids as well as nucleic acids that are derived from the expression vector, preferably using a method, such as is described in EP 0 714 987.

The pharmaceutical preparation may contain dosages of between 10-1000 μg/kg, more preferably between about 10-250 μg/kg, and most preferably between 10 and 75 μg/kg. Patients may be treated immediately upon presentation at the clinic with an inflammatory condition. Alternatively, patients may receive a bolus infusion every one to three hours, or if sufficient improvement is observed, a once daily infusion of the variant protein VII described herein.

B. Protein VII-Encoding Nucleic Acids

Protein VII-encoding nucleic acids may be used for a variety of purposes in accordance with the present invention. In a preferred embodiment of the invention, a nucleic acid delivery vehicle (i.e., an expression vector) for inflammation is provided wherein the expression vector comprises a nucleic acid sequence coding for a functional fragment of Protein VII as described herein. Administration of protein VII-encoding expression vectors to a patient results in the expression of protein VII polypeptide which serves to alter the inflammatory cascade. In accordance with the present invention, a protein VII peptide encoding nucleic acid sequence may encode a protein VII polypeptide as described herein whose expression reduces inflammation. In a preferred embodiment, a protein VII nucleic acid sequence encodes a human protein VII polypeptide variant and includes a secretory signal peptide.

Expression vectors comprising variant protein VII nucleic acid sequences may be administered alone, or in combination with other molecules useful for modulating inflammation. According to the present invention, the expression vectors or combination of therapeutic agents may be administered to the patient alone or in a pharmaceutically acceptable, or biologically compatible compositions.

In a preferred embodiment of the invention, the expression vector comprising nucleic acid sequences encoding the protein VII peptide variant is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors.

In a preferred embodiment of the present invention, methods are provided for the administration of a viral vector comprising nucleic acid sequences encoding a functional fragment of Protein VII. Adenoviral vectors of utility in the methods of the present invention preferably include at least the essential parts of adenoviral vector DNA. As described herein, expression of a protein VII polypeptide following administration of such an adenoviral vector serves to modulate inflammation.

Recombinant adenoviral vectors have found broad utility for a variety of gene therapy applications. Their utility for such applications is due largely to the high efficiency of in vivo gene transfer achieved in a variety of organ contexts.

Adenoviral particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, adenoviruses are known to infect a wide variety of cell types in vivo and in vitro by receptor-mediated endocytosis. The use of adenoviral vectors for this purpose is relatively safe as infection with adenovirus leads to a minimal disease state in humans comprising mild flu-like symptoms.

Due to their large size (˜36 kilobases), adenoviral genomes are well suited for use as gene therapy vehicles because they can accommodate the insertion of foreign DNA following the removal of adenoviral genes essential for replication and nonessential regions. Such substitutions render the viral vector impaired with regard to replicative functions and infectivity. Of note, adenoviruses have been used as vectors for gene therapy and for expression of heterologous genes.

For a more detailed discussion of the use of adenovirus vectors utilized for gene therapy, see Berkner, 1988, Biotechniques 6:616-629 and Trapnell, 1993, Advanced Drug Delivery Reviews 12:185-199.

It is desirable to introduce a vector that can provide, for example, multiple copies of a desired gene and hence greater amounts of the product of that gene. Improved adenoviral vectors and methods for producing these vectors have been described in detail in a number of references, patents, and patent applications, including: Mitani and Kubo (2002, Curr Gene Ther. 2(2):135-44); Olmsted-Davis et al. (2002, Hum Gene Ther. 13(11):1337-47); Reynolds et al. (2001, Nat Biotechnol. 19(9):838-42); U.S. Pat. No. 5,998,205 (wherein tumor-specific replicating vectors comprising multiple DNA copies are provided); U.S. Pat. No. 6,228,646 (wherein helper-free, totally defective adenovirus vectors are described); U.S. Pat. No. 6,093,699 (wherein vectors and methods for gene therapy are provided); U.S. Pat. No. 6,100,242 (wherein a transgene-inserted replication defective adenovirus vector was used effectively in in vivo gene therapy of peripheral vascular disease and heart disease); and International Patent Application Nos. WO 94/17810 and WO 94/23744.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for the expression of functional fragments of protein VII. For example, an El deleted type 5 adenoviral vector comprising nucleic acid sequences encoding protein VII under the control of a cytomegalovirus (CMV) promoter may be used to advantage in the methods of the present invention.

C. Pharmaceutical Compositions

The expression vectors of the present invention may be incorporated into pharmaceutical compositions that may be delivered to a subject, so as to allow production of a biologically active protein (e.g., a functional fragment of protein VII or derivative thereof). In a particular embodiment of the present invention, pharmaceutical compositions comprising sufficient genetic material to enable a recipient to produce a therapeutically effective amount of a protein VII polypeptide can influence inflammation in the subject. Alternatively, as discussed above, an effective amount of the variant protein VII polypeptide may be directly infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., anti-inflammatory agents) which influence inflammatory pathways.

In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of protein VII-fragment containing vectors or polypeptides, such labeling would include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques and guidance provided in the present invention. Therapeutic doses will depend on, among other factors, the age and general condition of the subject, the severity of the inflammatory phenotype, and the strength of the control sequences regulating the expression levels of the protein VII polypeptide fragment. Thus, a therapeutically effective amount in humans will fall in a relatively broad range that may be determined by a medical practitioner based on the response of an individual patient to vector-based protein VII treatment.

D. Administration

The variant protein VII polypeptides, alone or in combination with other agents may be directly infused into a patient in an appropriate biological carrier as described hereinabove. Expression vectors of the present invention comprising nucleic acid sequences encoding protein VII, or functional fragments thereof, may be administered to a patient by a variety of means to achieve and maintain a prophylactically and/or therapeutically effective level of the protein VII polypeptide. One of skill in the art could readily determine specific protocols for using the protein VII encoding expression vectors of the present invention for the therapeutic treatment of a particular patient. Protocols for the generation of adenoviral vectors and administration to patients have been described in U.S. Pat. Nos. 5,998,205; 6,228,646; 6,093,699; 6,100,242; and International Patent Application Nos. WO 94/17810 and WO 94/23744 which are also incorporated herein by reference in their entirety.

Protein VII peptides encoding adenoviral vectors of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. A clinician specializing in the treatment of patients with inflammatory disorders may determine the optimal route for administration of the adenoviral vectors comprising protein VII peptide encoding nucleic acid sequences based on a number of criteria, including, but not limited to: the condition of the patient and the purpose of the treatment.

The present invention also encompasses AAV vectors comprising a nucleic acid sequence encoding a protein VII polypeptide fragment.

Also provided are lentivirus or pseudo-typed lentivirus vectors comprising a nucleic acid sequence encoding a protein VII polypeptide fragment.

Also encompassed are naked plasmid or expression vectors comprising a nucleic acid sequence encoding a protein VII polypeptide fragment.

The following example is provided to illustrate various embodiments of the present invention. The example is illustrative and is not intended to limit the invention in any way.

### Example

### Materials and Methods Cells

Primary small airway epithelial cells (SAECs), U2OS, HeLa, 293, THP-1 and A549 cells were obtained from the American Tissue Culture Collection (ATCC) and grown according to the provider's instructions. Acceptor cells for generation of inducible cell lines were used as reported (Khandelia et al. (2011) Proc. Natl. Acad. Sci., 108:12799-12804). Protein VII, preVII and V were cloned from genomic DNA isolated from HeLa cells infected with adenovirus type 5 and inserted into the inducible plasmid cassette with a C-terminal HA tag using restriction enzymes BsrGI and AgeI. Positive clones were selected in DH5a cells, sequenced, and transfected into A549, U2OS or HeLa acceptor cells along with plasmid expressing the Cre recombinase. Recombined clones were selected by puromycin resistance (1 μg/mL) and induced with doxycycline (0.2 μg/mL) to express the desired protein. Protein expression was verified by immunofluorescence and western blot. All figures shown are after 4 days of induction unless otherwise stated. Protein VII and preVII were also verified by HPLC purification and mass spectrometry analysis. Point mutations were generated by gene synthesis from Genewiz.

### Viruses and Infections

Wild-type adenovirus type 5 (Ad5), adenovirus type 9 (Ad9), adenovirus type 12 (Ad12), and recombinant adenovirus vectors expressing only GFP were propagated in 293 cells as described (Kozarsky et al. (1996) Nat. Genet., 13:54-62). Recombinant adenovirus vector with VII-GFP replaced in the El region was obtained (Orazio et al. (2011) J. Virol., 85:1887-1892). Infections were carried out as described (Le et al. (2006) Virology 351:291-302) using a multiplicity of infection of 10 for primary cells and cell lines for Ad5 infections. Ad9 and Ad12 infections were carried out with a multiplicity of infection of 50 and 20, respectively. Ad5-flox-VII was prepared using standard methods in 293 cells. LoxP sites were added flanking protein VII in the Ad5 genome resulting in protein VII deletion during infection of 293 cells expressing Cre recombinase.

Antibodies Primary antibodies were purchased from Covance (HA MMS-101R), Abcam (H1 ab4269, H3 ab1791, HMGB1 ab18256, HMGB2 ab67282), Millipore (H2A 07-146, prosurfactin-C AB3786), and Santa Cruz (Ku86 sc5280, tubulin sc69969). The antibodies to DBP, adenoviral late proteins, terminal protein and protein VII were also obtained (Kozarsky et al. (1996) Nat. Genet., 13:54-62; Reich et al. (1983) Virology 128:480-484). Secondary antibodies for immunoblotting were obtained from Jackson ImmunoResearch and secondary antibodies for immunofluorescence were obtained from Life Technologies.

### Immunofluorescence

Cells were grown on glass coverslips in 24-well plates and either infected or induced with doxycycline (0.2 μg/mL). Cells were harvested for immunofluorescence at the indicated time points, washed in phosphate-buffered saline (PBS), fixed in 4% paraformaldehyde for 15 minutes and post-fixed with 100% ice-cold methanol for 5 minutes. Coverslips were then blocked and stained as described (Lilley et al. (2011) PLoS Pathog., 7:e1002084) and mounted using ProLong Gold Antifade Reagent (Life Technologies). Immunofluorescence was visualized using a Zeiss LSM Confocal microscope and ZEN 2011 software. Images were processed using ImageJ and assembled with Adobe CS6. All scale bars show in the Figures are 10 μm unless otherwise stated.

### Immunoblotting

Western blot analysis was carried out using standard methods. Briefly, equal amounts of total protein lysates were separated by sodium dodecyl sulfate-polyacrylamide gel electrophoresis (SDS-PAGE) and transferred to a nitrocellulose membrane (Millipore) for at least 30 minutes at 30V. Membranes were stained with ponceau to confirm protein loading and blocked in 5% milk in TBST containing 0.1% azide. Membranes were incubated with primary antibodies overnight, washed for 30 minutes in TBST and incubated with secondary antibodies conjugated to horseradish peroxidase (Jackson Laboratories) for 1 hour. Membranes were washed again and proteins were visualized with Pierce ECL Western Blotting Substrate (Thermo Scientific) and detected using a Syngene G-Box.

### Mice

All mice were housed in SPF conditions. All studies in mice were carried out in accordance with the recommendations in the Guide for the Care and Use of Laboratory Animals of the National Institutes of Health. C57BL/6J male mice aged 8-10 weeks were used for experiments. Mice were sedated with ketamine and xylazine. Once sedated, mice underwent orotrachial intubation, as described (Das et al. (2013) J. Vis. Exp., (73):e50318), with a 20G angiocatheter from BD (Franklin Lakes, N.J.). Mice subsequently received 5e10 GC of recombinant adenovirus expressing VII-GFP or GFP purified as described above. Four days after infection, mice were exposed to aerosolized LPS, 3 mg/mL for 30 minutes as described (Jeyaseelan et al. (2004) Infect. Immun., 72:7247-7256). One day after LPS exposure, BAL, and lung tissue were harvested as detailed (Nick et al. (2000) J. Immunol., 164:2151-2159) and examined for HMGB1 content (ELISA, Chondrex 6010) and neutrophil count (hematoxylin and eosin stain kit EMD 65044/93). Immunostaining was carried out using standard methods. A minimum of four biological replicates were used for each condition studied. Mice were assigned a random number and color at the start of the experiment and were randomized. Technicians carrying out the experiments were blinded to the identity of the samples. Tissue samples were assigned a random study number such that the technician performing the analysis was blinded. Unblinding for the purpose of data analysis occurred only after all data had been collected.

### Salt Fractionation of Nuclei

Salt fractionation of nuclei was adapted from established protocols (Zaret, K. (2005) Micrococcal nuclease analysis of chromatin structure. Curr. Protoc. Mol., Biol. Chapter 21, Unit 21.1; Teves et al. (2012) Methods Mol. Biol., 833:421-432). Briefly, 2-4×10⁷ cells were collected and resuspended in 2 mL of ice-cold buffer I (0.32 M sucrose, 60 mM KCl, 15 mM NaCl, 5 mM MgCl₂, 0.1 mM EGTA, 15 mM Tris, pH 7.5, 0.5 mM DTT, 0.1 mM PMSF and protease inhibitor cocktail from Roche). To dissolve the plasma membrane, 2 mL ice-cold buffer I supplemented with 0.1% IGEPAL were added and samples were incubated on ice for 10 minutes. The 4 mL of nuclei was layered on 8 mL of ice-cold buffer II (1.2 M sucrose, 60 mM KCl, 15 mM NaCl, 5 mM MgCl₂, 0.1 mM EGTA, 15 mM Tris, pH 7.5, 0.5 mM DTT, 0.1 mM PMSF and protease inhibitor cocktail from Roche) and centrifuged for 20 minutes at 10,000×g and 4° C. The pelleted nuclei were resuspended in 400 μL buffer III (10 mM Tris pH 7.4, 2 mM MgCl₂, 0.1 mM PMSF) supplemented with 5 mM CaCl₂ and the DNA was digested to mononucleosomes by addition of 1 unit of MNase (Sigma-Aldrich, N3755). The reaction was incubated at 37° C. for 30 minutes and then stopped by addition of 25 μL of 0.1M EGTA. The samples were centrifuged for 10 minutes, 350×g, at 4° C., and supernatants were set aside for western blot analysis. The pellet was resuspended in 400 μL of buffer IV (70 mM NaCl, 10 mM Tris pH 7.4, 2 mM MgCl₂, 2 mM EGTA, 0.1% Triton X-100, 0.1 mM PMSF) with 80 mM salt and rotated for 30 minutes at 4° C. The sample was centrifuged for 10 minutes at 350×g, 4° C., and the supernatant collected for western blot analysis. This step was repeated for salt concentrations in buffer IV of 150 mM, 300 mM and 600 mM. The final pellet was resuspended in 400 μL ddH₂O and all samples were analyzed together by western blot. An aliquot of each supernatant was set aside for DNA purification using a PCR purification kit (Qiagen) and analyzed by agarose gel electrophoresis. Alternatively, 4×10⁷ cells were resuspended in 400 μL hypotonic buffer (10 mM HEPES pH=7.9, 1.5 mM MgCl₂, 10 mM KCl, 1:1000 PMSF, 0.5 mM DTT) and incubated on ice for 30 minutes. The cells were transferred to a 1 ml dounce tissue grinder and the cell membranes were gently disrupted with 40 strokes of a tight-fitting pestle. The samples were centrifuged for 5 minutes at 1,500 g and 4° C. The pelleted nuclei were resuspended in 400 μL buffer III and the fractionation was continued as described above.

### Preparation of Salt Fractions for Mass Spectrometry Analysis

All chemicals used for preparation of mass spectrometry samples were of at least sequencing grade and purchased from Sigma-Aldrich (St Louis, Mo.), unless otherwise stated. Only the 600 mM salt fraction was used for LC-MS/MS analysis. The 0.1% TritonX-100 detergent was removed from samples prior MS analysis by precipitation using chloroform (CHCl₃)-methanol (MeOH) precipitation (Wessel, et al. (1984) Anal. Biochem., 138:141-143). The protein pellet from CHCl₃-MeOH precipitation was resuspended in 6 M urea and 2 M thiourea in 50 mM ammonium bicarbonate. Samples were reduced with 10 mM DTT for 1 hour at room temperature and the carbamidomethylated with 20 mM iodoacetamide for 30 minutes at room temperature in the dark. After alkylation proteins were digested first with endopeptidase Lys-C (Wako, mass spectrometry grade) for 3 hours, after which the solution was diluted 10 times with 20 mM ammonium bicarbonate. Subsequently, samples were digested with trypsin (Promega) at an enzyme to substrate ratio of approximately 1:50 for 12 hours at room temperature. The samples were acidified with 5% formic acid (FA) to a pH≤3 and desalted using Poros Oligo R3 RP columns (PerSeptive Biosystems) packed in a P200 stage tip with C18 3M plug (3M Bioanalytical Technologies). Purified peptide samples were dried by lyophilization and stored at −20° C. until further analysis. This procedure was carried out for three biological replicas.

### Nano LC-MS/MS and Analysis of Salt Fractions

Samples were loaded onto a 16 cm C₁₈-AQ column (inner diameter 75 μm, 3 μm beads, Dr, Maisch GmbH, Germany) using an Easy nano-flow HPLC system (Thermo Fisher Scientific, Odense, Denmark). The nanoLC was coupled to an Orbitrap Fusion Tribrid Mass Spectrometer (Thermo Fisher Scientific, San Jose, Calif.) via a nanoelectrospray ion source (Thermo Fisher Scientific, San Jose, Calif.). Peptides were loaded in buffer A (0.1% formic acid) and eluted with a 120 minute linear gradient from 2-30% buffer B (95% acetonitrile, 0.1% formic acid). After the gradient, the column was washed with 90% buffer B. Mass spectra were acquired using a data-dependent acquisition method with the TopSpeed set with 3-second cycle. Spectra were acquired in the Orbitrap analyzer with mass range of 350-1200 m/z and 120,000 resolution (200 m/z), with a maximum injection time of 50 msec and an AGC target of 5×10e5. Signals with 2-5 charges were selected for HCD fragmentation using a normalized collision energy of 27, a maximum injection time of 120 msec and an AGC target of 10,000. Fragments were analyzed in the ion trap. Raw MS files were analyzed by MaxQuant (v1.5.2.8) (Cox et al. (2008) Nat. Biotechnol., 26:1367-1372) (www.maxquant.org). MS/MS spectra were searched against the UniProt-human database (Version June 2014, 59,345 entries). All used search parameters were default, with the exception of including the match between runs (1 minute window) and the iBAQ label-free quantification (Schwanhausser et al. (2011) Nature 473:337-342). The search included variable modifications of methionine oxidation and N-terminal acetylation, and fixed modification of carbamidomethyl cysteine. Each iBAQ value was log₂ transformed and subsequently normalized by the average protein abundance within each run. Biological process association analysis and process network enrichment were performed using the GeneGo's MetaCore pathways analysis package with false discovery rate (FDR)<5%; each GO term was ranked using p-value enrichment.

### Purification of Recombinant Protein VII-his

Protein VII was cloned from genomic DNA isolated from adenovirus infected HeLa cells into a pET21a backbone to generate a C-terminal hexahistidine tag. Positive clones were selected in DH5a cells, sequenced, and transformed into BL21 (DE3) cells (NEB C2527I). The purification of insoluble protein VII-His was adapted from existing protocols to purify histone proteins from E. coli (Tanaka et al. (2004) Methods 33:3-11; Luger et al. (1997) J. Mol. Biol., 272:301-311). Briefly, BL21 cells were inoculated from overnight cultures and grown to an optical density of 0.5-0.6 OD260, induced with 0.1 mM IPTG (Sigma) and harvested after 4 hours at 37° C. Cells pellets were resuspended in a mild buffer (50 mM Tris-HCl pH 8.0, 500 mM NaCl, 1 mM PMSF, 5% glycerol, 2.5 μg/mL aprotinin, leupeptin and pepstatin) and disrupted by sonication using a Branson 250 sonifier. The lysate was then centrifuged at 27,000×g for 20 minutes at 4° C. The supernatants were discarded and pellets were resuspended in a denaturing buffer (50 mM Tris-HCl, pH 8.0, 500 mM NaCl, 5% glycerol, 8 M urea). The suspension was centrifuged again to eliminate insoluble cell debris and the his-tagged protein was isolated using a cobalt resin (ThermoScientific 89964) according to the manufacturer's instructions for denaturing conditions. The purified protein was then dialyzed against water and lyophilized. Purified protein was verified by western blot and mass spectrometry.

### In Vitro Binding Assays

HMGB1-GST (Abnova) or GST (Sigma) were combined with recombinant protein VII-His at equimolar ratios and incubated at 4° C. for 1 hour. Complexes were then mixed with a cobalt resin (ThermoScientific 89964) to bind protein VII-His and any associated protein and washed three times in the binding buffer (50 mM Tris pH 8, 300 mM NaCl, 0.1% IGEPAL). The beads were then boiled in sample buffer, separated on a 4-12% NuPage gel and visualized by coomassie staining.

### Nucleosome In Vitro Binding and MNase Digestion Assays

Gel shift and MNase digestion assays were carried out as described (Falk et al. (2015) Science 348:699-703; Hasson et al. (2013) Nat. Struct. Mol. Biol., 20:687-695; Sekulic et al. (2010) Nature 467:347-351). Briefly, nucleosomes were reconstituted by incubating purified recombinant histones with ‘601’ DNA of either 195 or 147 bp over a series of dialysis. Recombinant protein VII-His was then combined with nucleosomes at various molar ratios, incubated at room temperature for 15 minutes, and analyzed by native gel electrophoresis. Complexes were also digested with MNase (Affymetrix) by addition of 1 unit per μg of DNA for 147 bp nucleosome experiments and 0.1 unit per μg of DNA for 195 bp nucleosome experiments, incubated at 22° C. for varying amounts of time followed by the addition of EGTA and guanidine thiocyanate to stop the reaction. The DNA fragments were then purified using a MinElute PCR purification kit (Qiagen) and analyzed on an Agilent 2100 Bioanalyzer as described (Falk et al. (2015) Science 348:699-703).

### Release Assay of HMGB1 in THP-1 Cells

THP-1 cells were seeded at a density of 2×10⁵ cells per well in a 24-well plate, and stimulated into macrophage-like cells by addition of 10 ng/mL PMA for 48 hours. Cells were washed in PBS and transduced with recombinant adenovirus vectors expressing only GFP or protein VII-GFP such that >90% of cells were GFP positive. At 48 hours post transduction, cells were washed and 200 μL of serum free RPMI was added. To stimulate the inflammasome, LPS (Sigma-Aldrich L2880) with a final concentration of 0.5 μg/mL was added to wells and incubated for 2 hours, then nigericin (Sigma-Aldrich N7143) was added with a final concentration of 10 μM for 1 hour. Supernatants were collected and proteins precipitated overnight at 4° C. with a final concentration of 20% trichloroacetic acid (Sigma), washed with acetone, dried, and resuspended in 1×sample buffer with reducing agent (Invitrogen). For ELISA analysis, supernatants were harvested directly and HMGB1 content was detected by the manufacturer's instructions (Chondrex 6010). Cells were also harvested by the addition of 1×sample buffer with reducing agent (Invitrogen) and boiled. Supernatants and lysates were analyzed together by western blot.

### Acid Extraction and Reverse Phase-HPLC for Purification of Protein VII and Analysis of Total Histone PTMs

Histones were prepared for mass spectrometry analysis as detailed (Kulej et al. (2015) Methods 90:8-20). Nuclei were isolated and histones from infected cells were extracted by acid as described (Lin et al. (2012) Meth. Enzymol., 512:3-28). The pre-protein VII and protein VII variants were fractionated using an offline RP-HPLC. Briefly, ˜100 μg proteins were resuspended in buffer A (0.1% 546 trifluoroacetic acid (TFA) in HPLC grade water) and loaded onto a C₁₈ 5 μm column (4.6 mm internal diameter×250 mm, Vydac) using a Beckman Coulter (System Gold, Brea, Calif.) HPLC (Buffer A: 0.1% TFA, Buffer B: 95% acetonitrile, 0.08% TFA). The proteins were separated using a gradient from 30-45% buffer B in 100 minutes at a flow-rate of 0.2 mL/minute. The fractions containing the proteins of interest were collected using an automatic fraction collector and individual peaks combined based on their UV signal. The fractions were subsequently dried by vacuum centrifugation and prepared for mass spectrometry (see below). Protein VII was purified from three biological replicates and analyzed as follows for MS.

### Mass Spectrometry Analysis of Protein VII PTMs

Sample Preparation/Protein VII:

RP-HPLC purified samples of protein VII variants were reduced in 10 mM dithiothreitol (DTT) in 50 mM ammonium bicarbonate for 1 hour at 56° C. After cooling to room temperature, samples were alkylated in 20 mM iodoacetamide in 50 mM ammonium bicarbonate for 30 minutes in the dark. Samples were digested with chymotrypsin or Arg-C, at an enzyme to substrate ratio of approximately 1:20 for 8 hours at 37° C. The samples were acidified to a final concentration of 5% formic acid to a pH≤3 and desalted using P200 stage tip columns packed with C₁₈3 M plug (3M Bioanalytical Technologies). Purified peptide samples were dried by lyophilization and stored at −20° C. until further analysis.

Nano LC-MS/MS Analysis of Histone PTMs:

The nanoLC-MS/MS analysis was performed as described (Kulej et al. (2015) Methods 90:8-20).

Nano LC-MS/MS Analysis of Protein VII Peptides:

The nanoLC-MS/MS analysis was performed in triplicate for each sample. Samples were loaded onto a 16 cm C18-AQ column (inner diameter 75 μm, 3 μm beads, Dr, Maisch GmbH, Germany) using an Easy nano-flow HPLC system (Thermo Fisher Scientific, Odense, Denmark). The nanoLC was coupled to an Orbitrap Velos Pro Mass Spectrometer (Thermo Fisher Scientific) via a nanoelectrospray ion source (Thermo Fisher Scientific, San Jose, Calif.). Peptides were loaded in buffer A (0.1% formic acid) and eluted with a 45 minute linear gradient from 2-30% buffer B (95% acetonitrile, 0.1% formic acid). After the gradient, the column was washed with 90% buffer B. Mass spectra were acquired using a data-dependent acquisition method with the Top15 most intense ions. Spectra were acquired in the Orbitrap analyzer with mass range of 350-1600 m/z and 60,000 resolution (400 m/z), with a maximum injection time of 10 msec and an AGC target of 10×10⁶. Signals above 1000 count charges were selected for HCD fragmentation using normalized collision energy of 36, a maximum injection time of 100 msec and an AGC target of 50,000. Fragments were analyzed in the orbitrap.

Data Processing of Protein VII Spectra:

Raw mass spectrometer files were analyzed using Proteome Discoverer (v1.4, Thermo Scientific, Bremen, Germany). MS/MS spectra were converted to .mgf files and searched against the UniProt-adenovirus C serotype 5 database using Mascot (v2.5, Matrix Science, London, UK). Database searching was performed with the following parameters: precursor mass tolerance 10 ppm; MS/MS mass tolerance 0.05 Da; enzyme chymotrypsin (Promega) or Arg-C(Roche), with two missed cleavages allowed; fixed modification was cysteine carbamidomethylation; variable modifications were methionine oxidation, serine/threonine/tyrosine phosphorylation, lysine acetylation and methylation, asparagine and glutamine deamidation. Specifically, phosphorylation, acetylation, and methylation were searched separately, not as co-existing modifications. Peptides were filtered for <1% false discovery rate, Mascot ion score >20 and peptide rank 1.

### Co-Immunoprecipitation of Protein VII-HA

A549 cells were induced to express protein VII with doxycycline for four days as described above. Approximately 4×10⁷ cells were harvested and pelleted for each immunoprecipitation reaction. Cell pellets were resuspended in 500 μl of IC wash buffer with protease inhibitors (20 mM HEPES pH 7.9, 110 mM KOAc, 2 mM MgCl₂, 150 mM NaCl, 0.1% Tween-20, 0.1% Triton X) and incubated on ice for 10 minutes with intermittent vortexing to disrupt cells. Samples were then incubated on ice for 1 hour with 5 μl of benzonase (Millipore) added to each sample to digest DNA to ˜150 bp, which was confirmed by DNA isolation and agarose gel analysis. Samples were then sonicated in a Diagenode Bioruptre for 30 seconds on and 30 seconds off for five rounds at 4° C. and centrifuged at 14,000 g for 15 minutes at 4° C. Supernatants were then incubated rotating for 1 hour at 4° C. with 30 μl of HA-conjugated magnetic beads (Thermo Scientific) and washed three times for five minutes in IC buffer. Isolated proteins were eluted with 100 μl of 2 mg/ml HA peptide (Thermo Scientific) for 20 minutes rotating at 37° C. and separated on and SDS-PAGE gel. For protein separation by SDS-PAGE the NuPAGE IDE System was used (NuPAGE Novex 4-12% bis-tris 1.0 mm gels, Invitrogen, USA). Uninduced cells were used as a negative control. The immunopreciptation was carried out in biological triplicate and pull-down of protein VII-HA and HMGB1 was confirmed by western blotting standard techniques as described above.

### Quantitative PCR

Genomic DNA was isolated using the PureLink Genomic DNA kit (Thermo Scientific). Quantitative PCR was performed using primers specific for viral DBP (5′gccattgcgcccaagaagaa; (SEQ ID NO: 16) and 5′ ctgtccacgattacctctggtgat; (SEQ ID NO: 17), protein VII (5′gcgggtattgtcactgtgc; SEQ ID NO: 18) and 5′ cacccaatacacgttgccc; SEQ ID NO: 19), and cellular tubulin (5′ccagatgccaagtgacaagac; SEQ ID NO: 20 and 5′ gagtgagtgacaagagaagcc; SEQ ID NO: 21). Values for DBP and VII were normalized internally to tubulin and to the 4 hour time point to control for any variation in virus input. RNA was isolated using the RNeasy Mini Kit (Qiagen) and reverse transcribed using the High Capacity RNA to cDNA Kit (Applied Biosystems). Quantitative PCR was performed using primers specific for HMGB1 (5′taactaaacatgggcaaaggag; SEQ ID NO: 22 and 5′ tagcagacatggtcttccac; SEQ ID NO: 23) and beta actin (5′gcaccacaccttctacaatgag; SEQ ID NO: 24 and 5′ ggtctcaaacatgatctgggtc; SEQ ID NO: 25). Quantitative PCR was performed using the standard protocol for Sybr Green (Thermo Scientific) and analyzed using the ViiA 7 Real-Time PCR System (Thermo Scientific).

### Precision Cut Lung Slice (PCLS) Immunofluorescence

PCLS were obtained and prepared as described (Cooper et al. (2008) J. Allergy Clin. Immunol., 122:734-740; Koziol-White et al. (2011) Expert Rev. Respir. Med., 5:767-777). De-identified human lung tissue from donors was obtained from the National Disease Research Interchange (NDRI), Philadelphia, Pa. Samples were infected with 10⁸ pfu of Ad5 per slice or 10⁹ GC of rAd VII-GFP for 24 hours. Samples were fixed in 4% PFA at room temperature for 15 minutes and washed three times in PBS. Samples were permiabilized with 0.5% Triton X and washed twice more in PBS. Samples were then incubated with 3% BSA and 0.03% Triton X in PBS for 1 hour to block. Primary antibodies (DBP or HMGB1) were incubated in the same buffer for 1 hour and then samples were washed three times in PBS with 3% BSA, incubated with secondary antibodies and DAPI for 1 hour, and washed three more times. Whole slices were mounted on slides with mounting solution and imaged by confocal microscopy.

Fluorescence Recovery after Photobleaching (FRAP)

Full-length HMGB1 was cloned from pcDNA3.1 Flag-hHMGB1 (Addgene 31609) into pEGFP-N1 containing a L221K mutation to prevent dimerization of GFP molecules (Zacharias et al. (2002) Science 296:913-916). A549 cells were induced to express protein VII for four days with doxycycline in glass-bottom dishes. Cells were then transfected with the construct that constitutively expresses HMGB1 with a monomeric GFP C-terminal tag. FRAP was carried out using standard methods on a Zeiss LSM confocal microscope. Diffusion coefficients were calculated using the “simFRAP” algorithm (imagej.nih.gov/ij/plugins/simfrap/index.html), a simulation based approach to FRAP analysis (Blumenthal et al. (2015) Sci. Rep., 5:11655).

### Statistical Analyses

Statistical details are reported in each figure legend. Statistical analyses were performed on at least three different biological replicates, unless otherwise stated in the figure legend. The sample size was chosen to provide enough statistical power to apply parametric tests (one- or two-tailed homoscedastic t-test). The t-test was considered as valuable statistical test since binary comparisons were performed and the number of replicates was limited. Furthermore, the homoscedastic t-test was applied assuming that the variance between the two datasets would remain homogeneous due to the use of the same cell lines in culture with and without protein VII expression. No samples were excluded as outliers (this applies to all proteomics analyses described). Proteins with p-value smaller than 0.05 were considered as significantly altered between the two tested conditions for two-tailed and one-tailed t-test. Data distribution was assumed to be normal. The nanoLC-MS analysis was performed in triplicate for each sample to determine technical variation. All proteomics raw files generated for this manuscript are collected into the public database Chorus (chorusproject.org/, Project number: 1047).

### Results

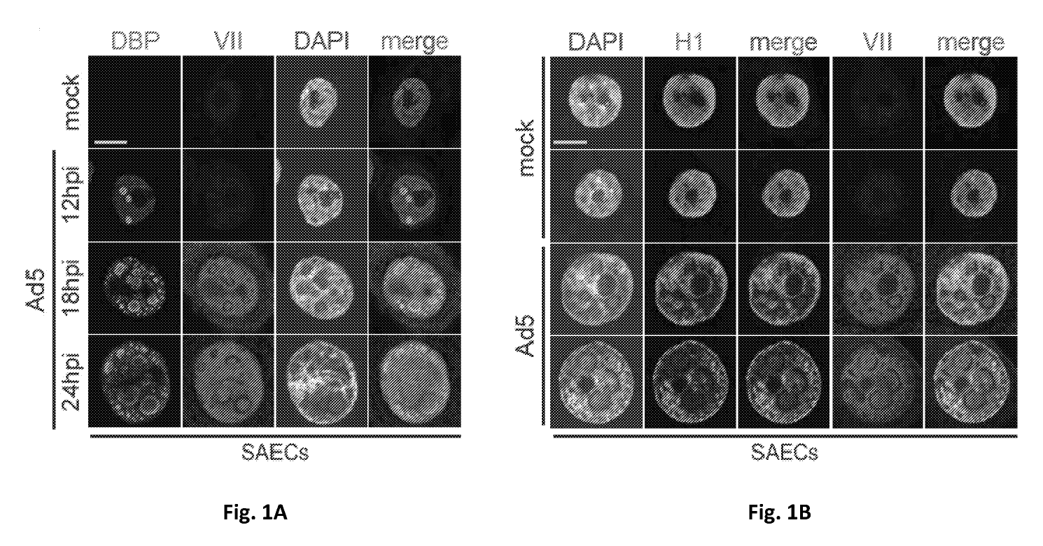

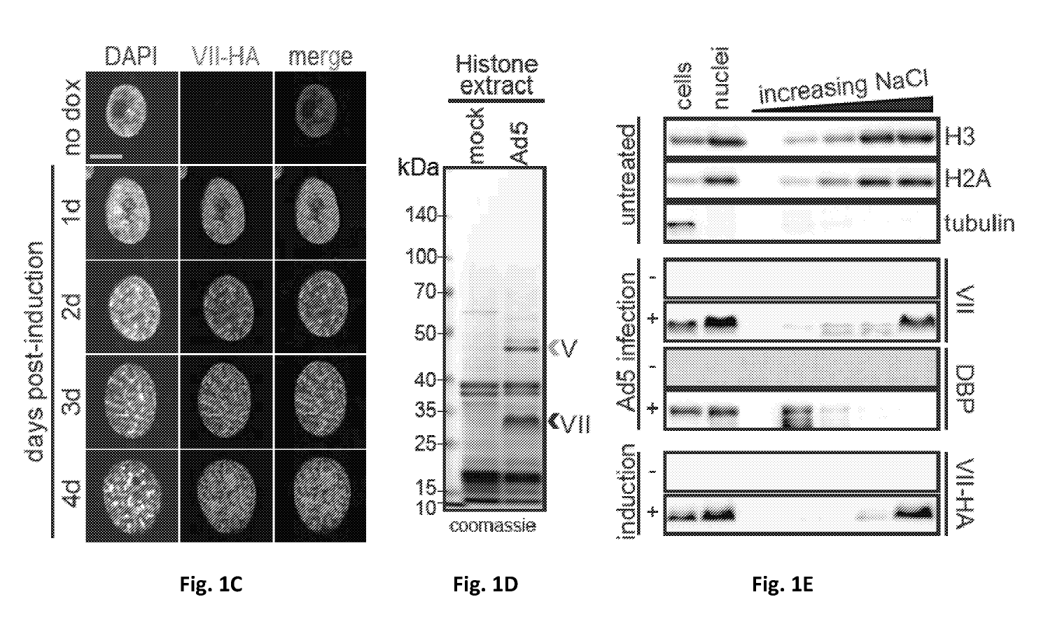

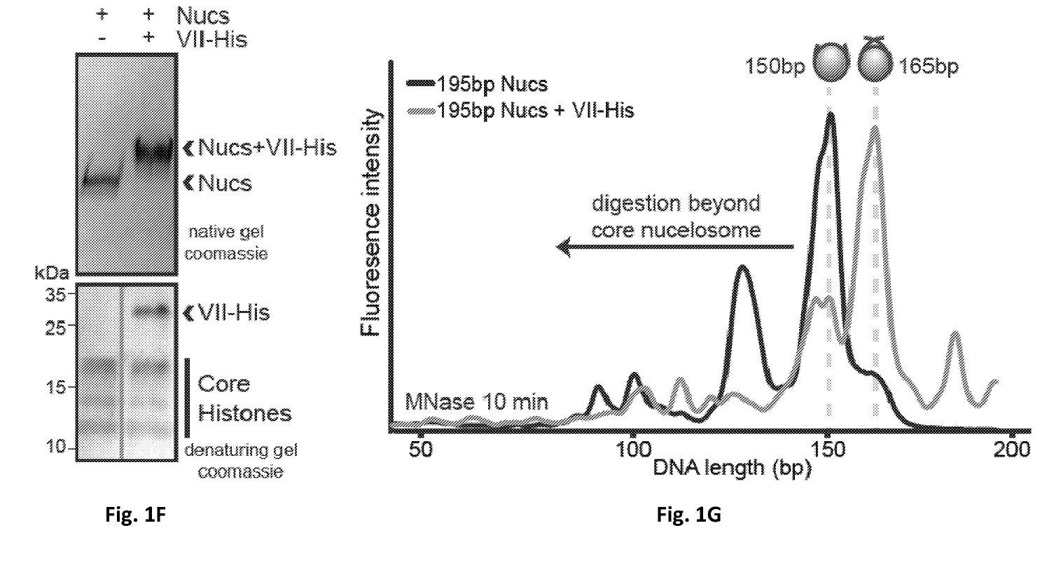

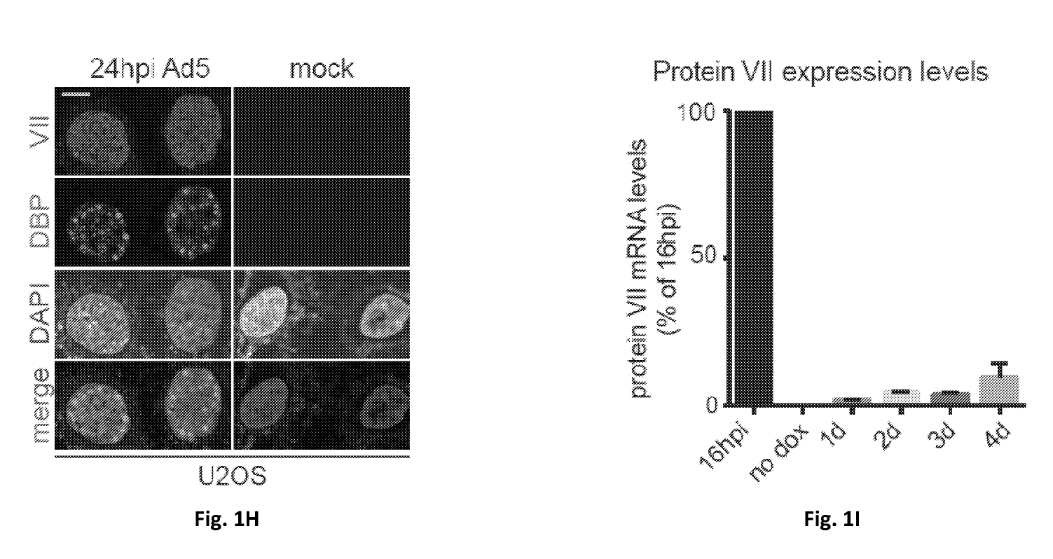

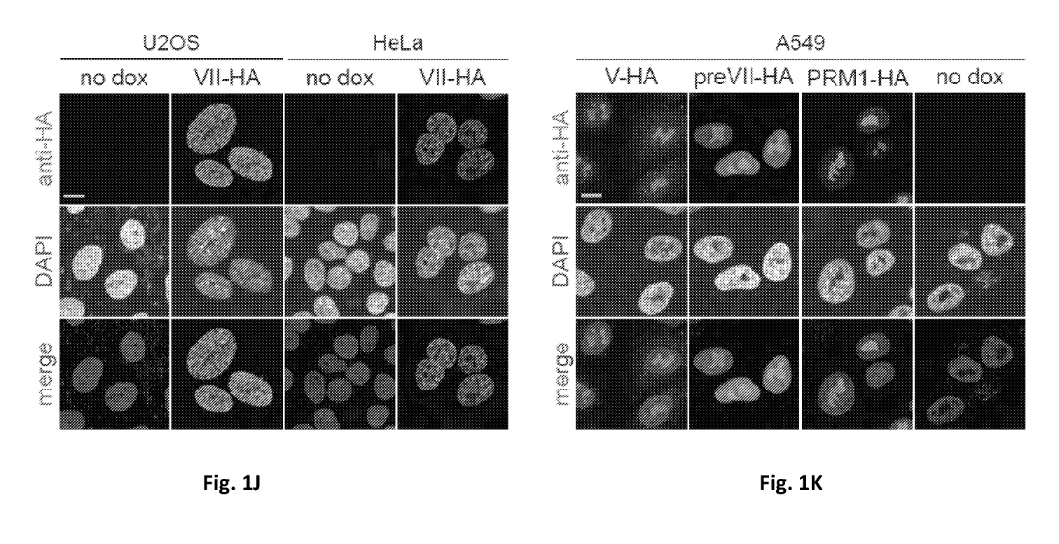

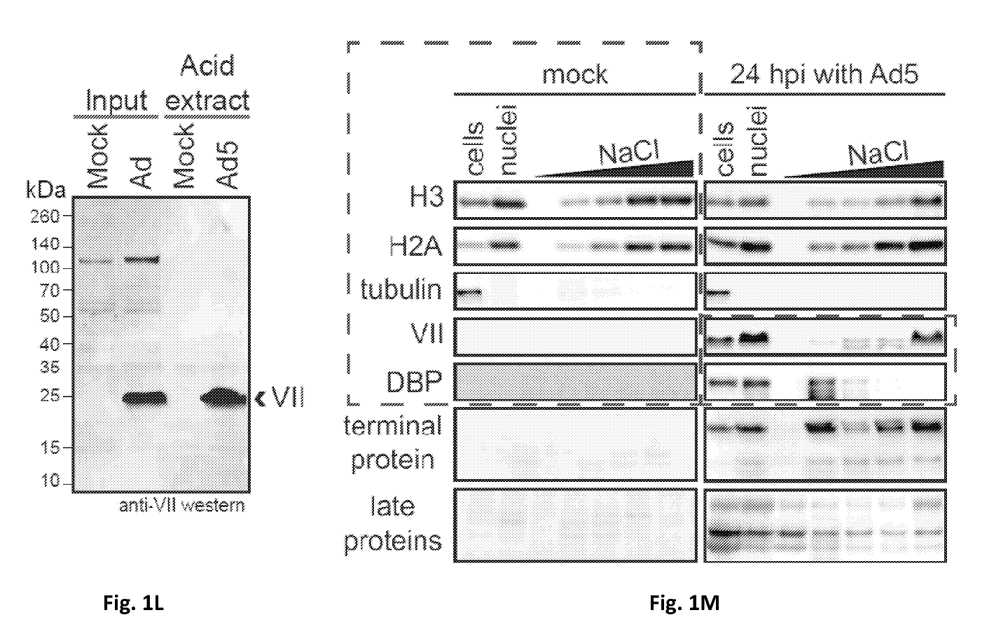

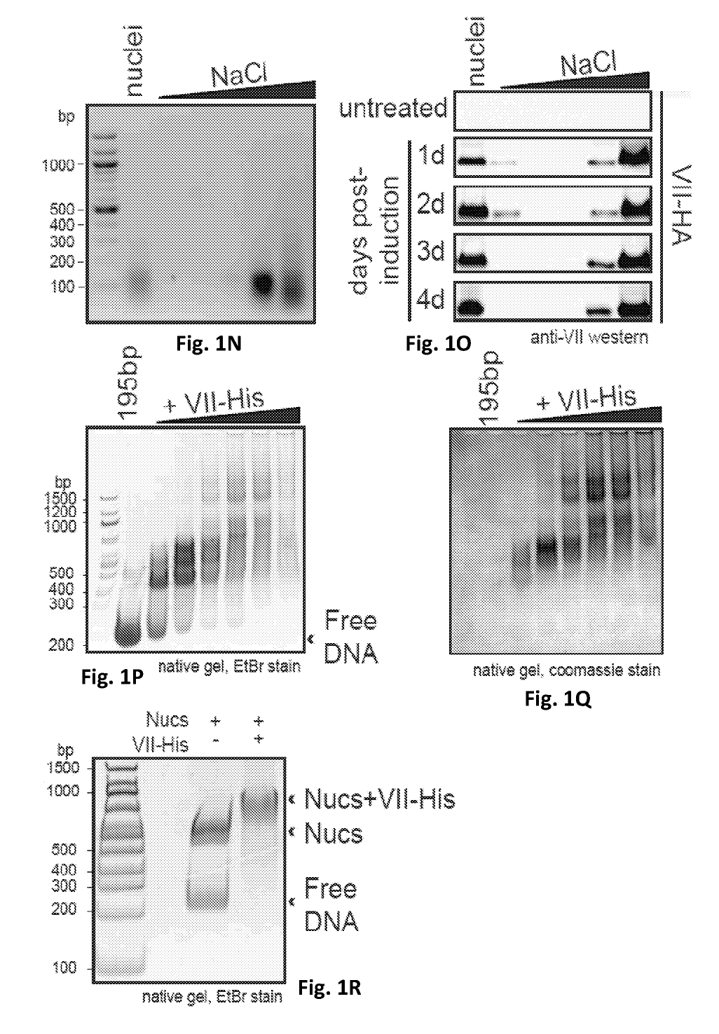

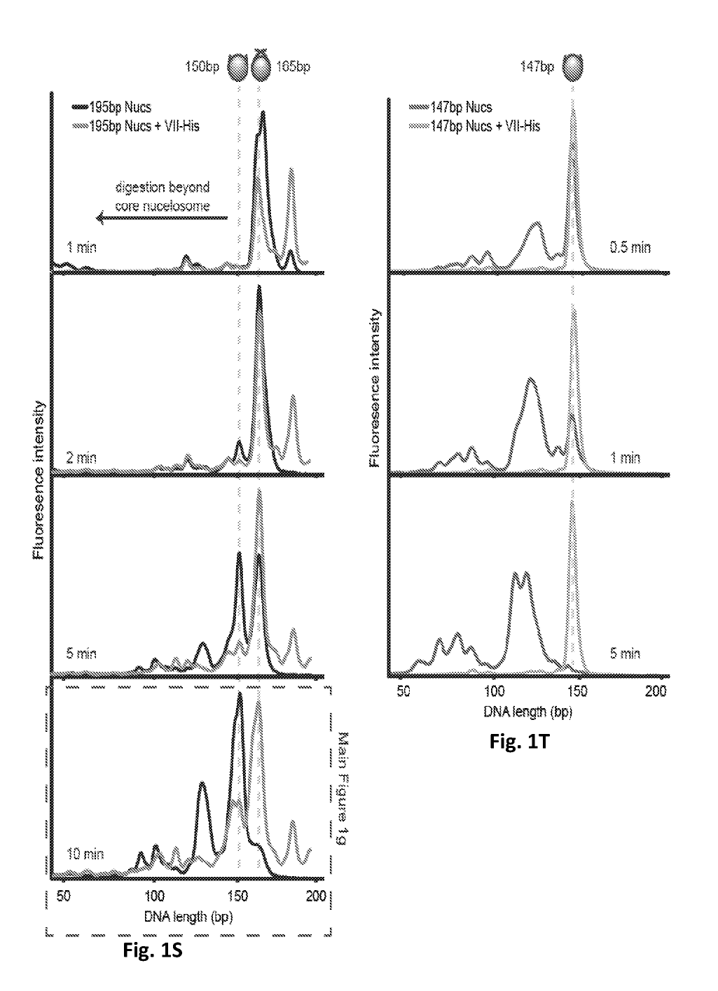

As viruses commandeer cellular functions to promote viral production, they induce numerous cellular changes. Manipulation of host chromatin is important for viral takeover of cellular functions (Elde et al. (2009) Nat. Rev. Microbiol., 7:787-797; Paschos et al. (2010) Trends Microbiol., 18:439-447; Marazzi et al. (2012) Nature 483:428-433; Ferrari et al. (2009) Nat. Rev. Genet., 10:290-294; Knipe et al. Virology 435:141-156). Although, there are known examples of viral control by manipulating gene expression (Smale et al. (2014) Annu. Rev. Immunol., 32:489-511; Marazzi et al. (2012) Nature 483:428-433; Ferrari et al. (2014) Cell Host Microbe 16:663-676), an alternative strategy for immune evasion could exploit cellular chromatin to impact extracellular signaling. Genomes of DNA viruses are compacted and packaged into virus particles with small basic proteins encoded by host or virus. Adenoviruses encode protein VII, a small basic protein packaged with viral genomes (Lischwe et al. (1977) Nature 267:552-554; Chatterjee et al. (1986) EMBO J 5:1633-1644; Vayda et al. (1983) Nuc. Acids Res., 11:441-460). Here, it was hypothesized that protein VII contributes to host chromatin manipulation. Protein VII localization was investigated during infection, and it was found present at both viral replication centers stained for viral DNA binding protein DBP (FIGS. 1A, 1H), and cellular chromatin stained for histone H1 and DAPI (FIG. 1B). These observations suggest protein VII functions on both viral and host genomes. To determine protein VII's impact on cellular chromatin, cell lines were generated with inducible expression. In multiple cell types, protein VII accumulation altered nuclear DNA into a punctate appearance (FIGS. 1C, 1I, 1J). It was then tested whether other basic proteins produce similar effects on chromatin. Viral core protein V and the precursor of protein VII (preVII) localized to nucleoli and did not affect chromatin appearance (FIG. 1K). Human protamine PRM1, a basic protein involved in sperm DNA compaction (Wykes et al. (2003) J. Biol. Chem., 278:29471-29477), also localized to nucleoli and did not affect chromatin appearance (FIG. 1K). Taken together, the data demonstrate that protein VII is sufficient to alter cellular chromatin and is distinct from other small basic proteins.

To affect cellular chromatin at the nucleosome level during infection, it was reasoned that protein VII must be abundant and associated with histones. Acid extraction of histones (Lin et al. (2012) Meth. Enzymol., 512:3-28; Shechter et al. (2007) Nat. Protocols 2:1445-1457) from infected cells, revealed viral proteins VII and V isolated with cellular histones (FIG. 1D), as verified by western blot (FIG. 1L) and mass spectrometry. Protein VII abundance was comparable to cellular histone levels (FIG. 1D). Association of protein VII with cellular chromatin was further analyzed by salt fractionation of nuclei (Teves et al. (2012) Methods Mol. Biol., 833:421-432). Protein VII was found with cellular histones and DNA in high salt fractions (FIGS. 1E, 1M, 1N, 1O). Ectopically expressed protein VII is also found in high salt fractions, in contrast to other viral proteins that elute at low salt (FIGS. 1E and 1M). These data indicate that protein VII is highly abundant and tightly associated with cellular chromatin.

It was hypothesized protein VII interacts with chromatin by forming complexes with DNA, histones, or nucleosomes, and protein VII interactions were examined in vitro. Purified recombinant protein VII binds to DNA (FIGS. 1P, 1Q). Nucleosomes were reconstituted in vitro with recombinant histone proteins on 195 base pairs (bp) of DNA (Falk et al. (2015) Science 348:699-703). Protein VII changed nucleosome mobility upon native gel electrophoresis (FIG. 1F, 1R). Native gel bands were analyzed by denaturing SDS-PAGE, and confirmed complexes contained core histones with protein VII (FIG. 1F, bottom). Unlike protamines (Wykes et al. (2003) J. Biol. Chem., 278:29471-29477), protein VII forms complexes with nucleosomes but does not appear to replace histones. Next, it was examined whether protein VII association with nucleosomes affects DNA wrapping using microccocal nuclease (MNase) digestion followed by DNA fragment analysis (Falk et al. (2015) Science 348:699-703). Protein VII pauses nucleosomal DNA digestion at ˜165 bp, the point at which DNA strands crossover the nucleosome dyad (FIGS. 1G, 1S). In contrast, nucleosome digestion alone paused with core particles at ˜150 bp, indicating protein VII encumbers DNA access. Unlike linker histone binding that is dependent on DNA length (White et al. (2016) Sci. Rep. 6:19122), protein VII protects against MNase digestion on the nucleosome core particle of 147 bp (FIG. 1T). Protein VII alone protects DNA from MNase digestion, as would be expected given its role in the viral core. Together, these data demonstrate protein VII binds directly to nucleosomes and limits DNA accessibility at the DNA entry/exit site.

Post-translational modifications (PTMs) on histones are central to regulating chromatin structure (Lin et al. (2012) Meth. Enzymol., 512:3-28; Kouzarides, T. (2007) Cell 128:693-705). Due to the histone-like nature of protein VII (Lischwe et al. (1977) Nature 267:552-554), it was hypothesized it is subject to post-translational modification similar to histones. Protein VII precursor may be acetylated by amino-terminal addition during protein synthesis (Fedor et al. (1980) J. Virol., 35:637-643). It was noted that protein VII contains conserved lysine residues within an AKKRS (SEQ ID NO: 26) motif (Robinson, C. M. et al. (2013) Sci. Rep., 3:1812), similar to the commonly modified canonical histone motif ARSK (Kouzarides, T. (2007) Cell 128:693-705). Therefore, protein VII was purified from histone extracts over an adenovirus infection time course by reverse phase HPLC (FIGS. 2A, 2D, 2E, 2F). Consistent with observations from histone extracts (FIG. 1D), protein VII levels were comparable to endogenous histones. Purified protein VII and preVII were digested with chymotrypsin to distinguish the two proteins, and peptides were analyzed by tandem mass spectrometry. Several PTMs were identified, with two acetylation sites and three phosphorylation sites as most abundant (FIG. 2B, 2G, 2I). Interestingly, acetylation sites were identified on ectopically expressed protein VII but not on protein VII in virus particles (FIG. 2H). This may provide a mechanism for distinguishing protein VII bound to cellular chromatin from protein destined for packaged virus. To investigate relevance of identified PTMs, modified sites were mutated in protein VII. An alanine-replacement mutant for all five PTM sites localized to nucleoli instead of cellular chromatin (FIG. 2C). Results with individual point mutations indicate the K3 residue is important for chromatin localization, and employing glutamine as an acetylation mimic (K3Q) mirrored the pattern of wild-type protein (FIG. 2C). Effects induced by protein VII are not due to global alteration of histone PTMs since only six PTMs on histones H3 and H4 showed minor but significant changes (FIGS. 2J-2L). These data indicate protein VII modification plays critical roles during virus infection.

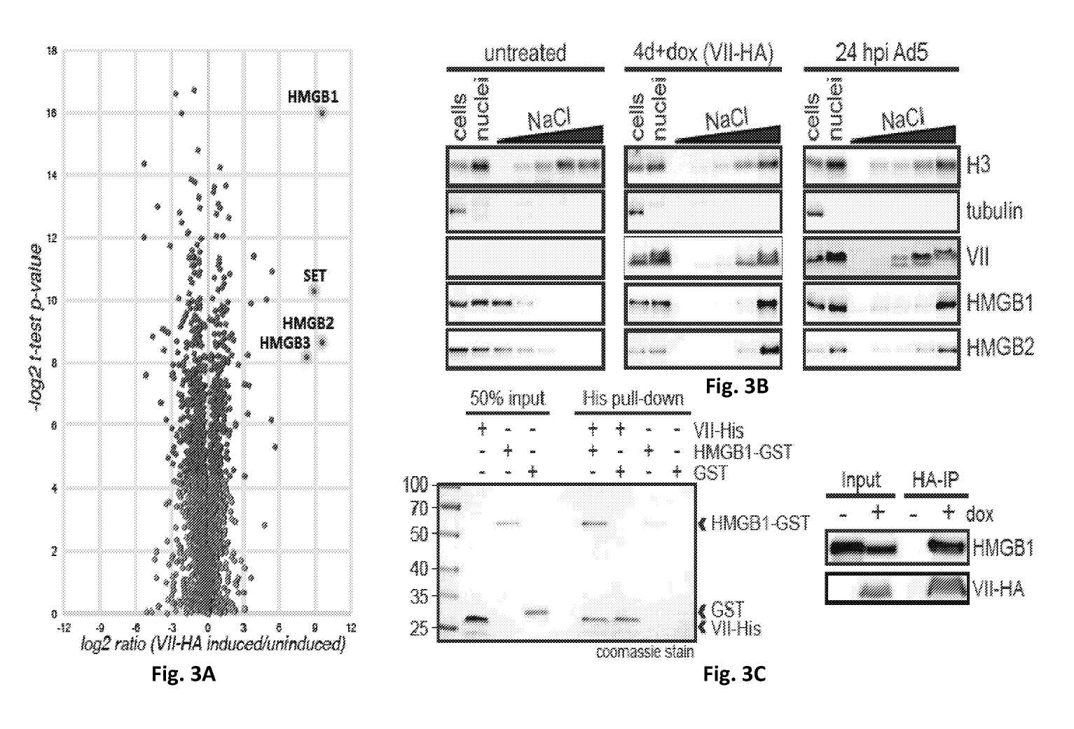

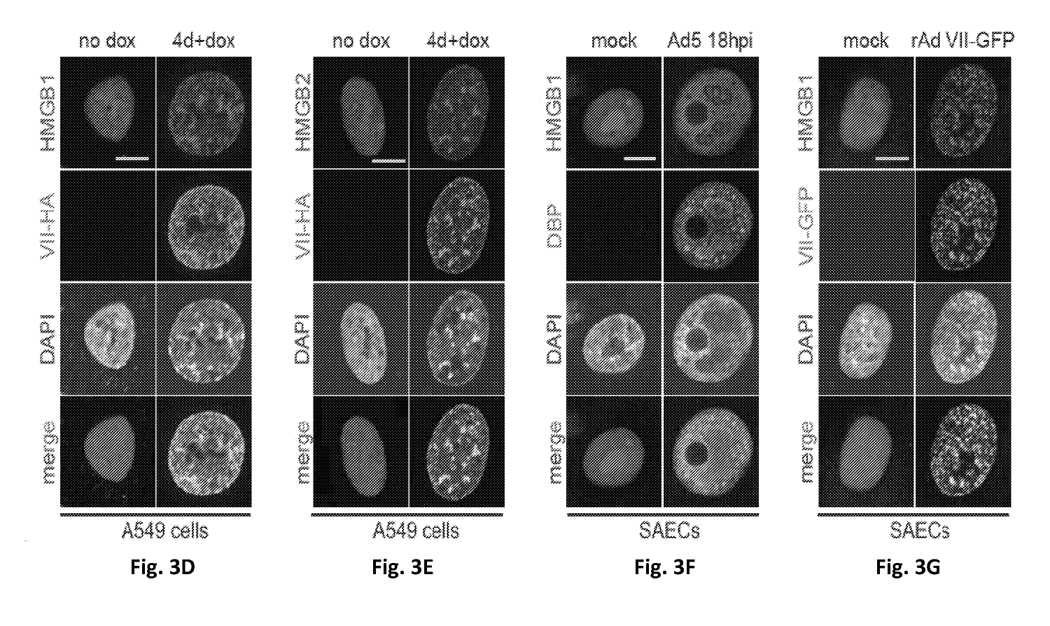

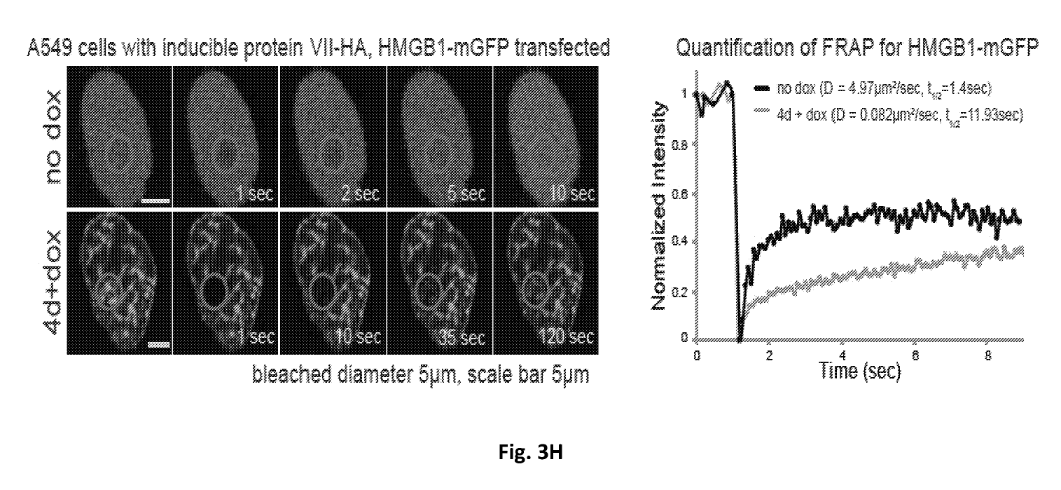

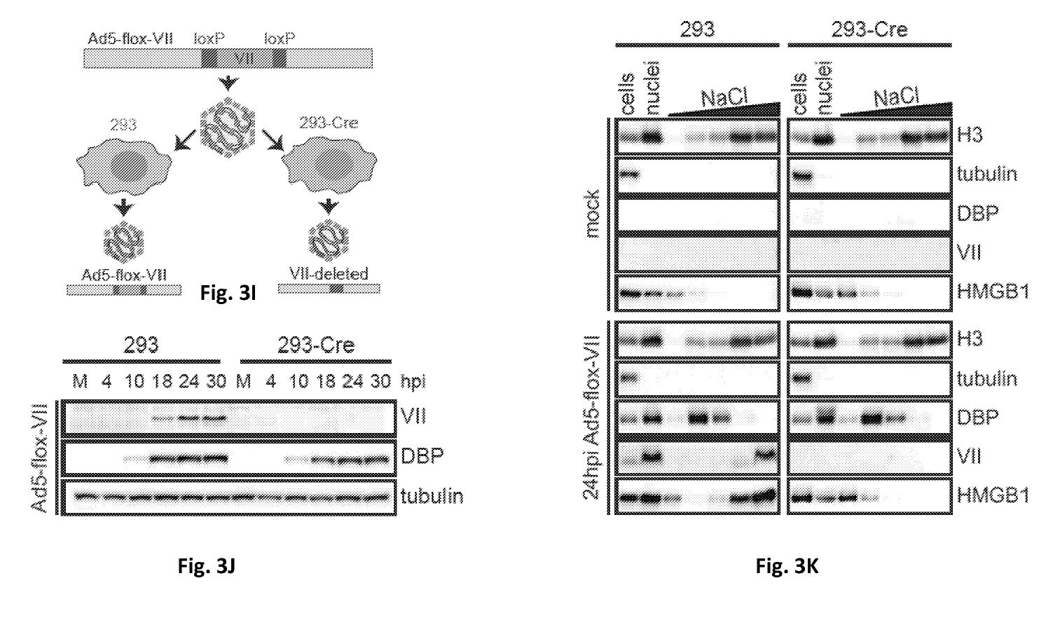

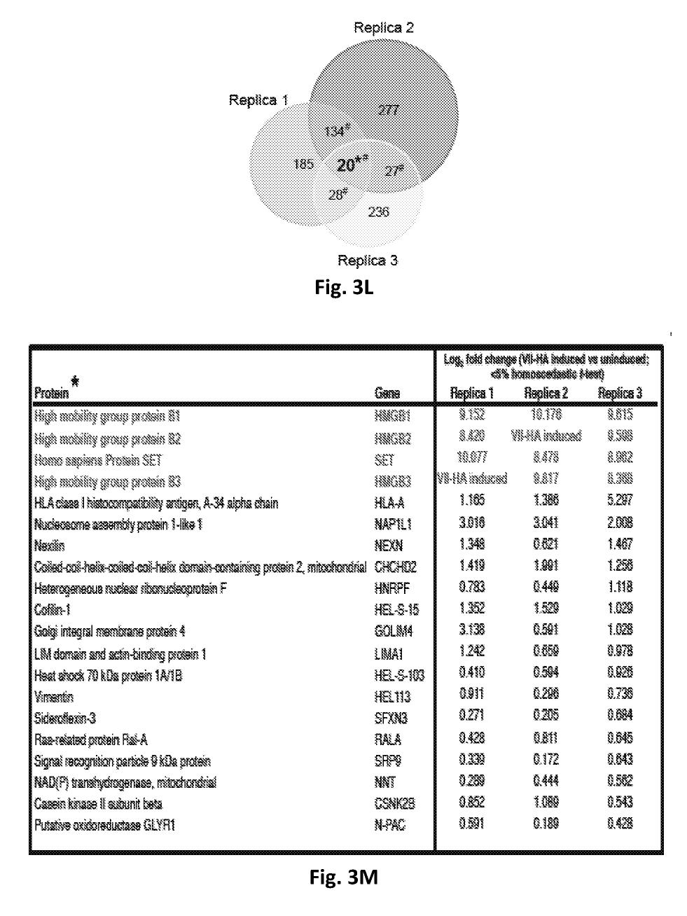

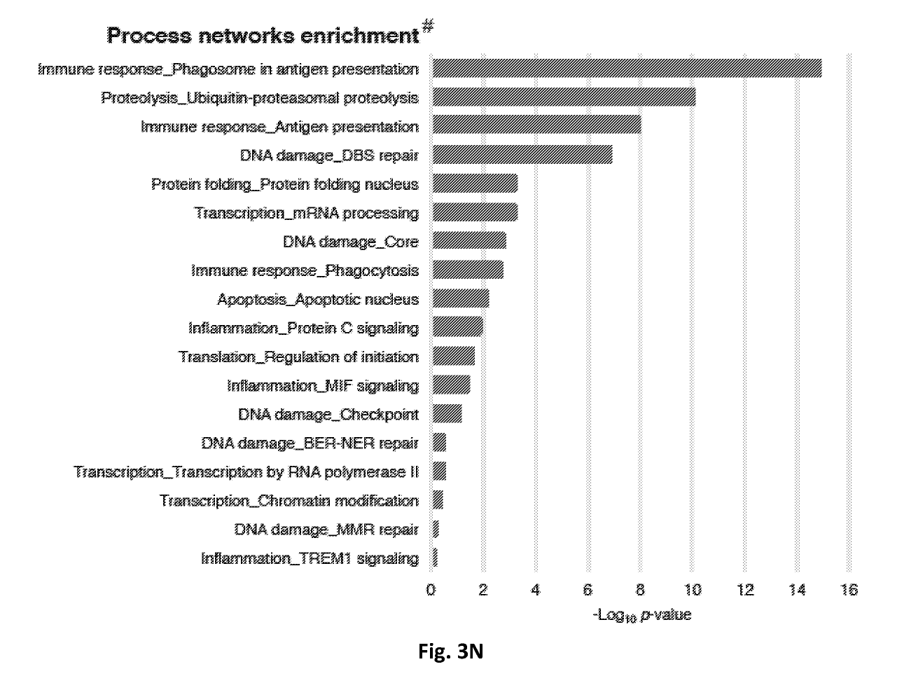

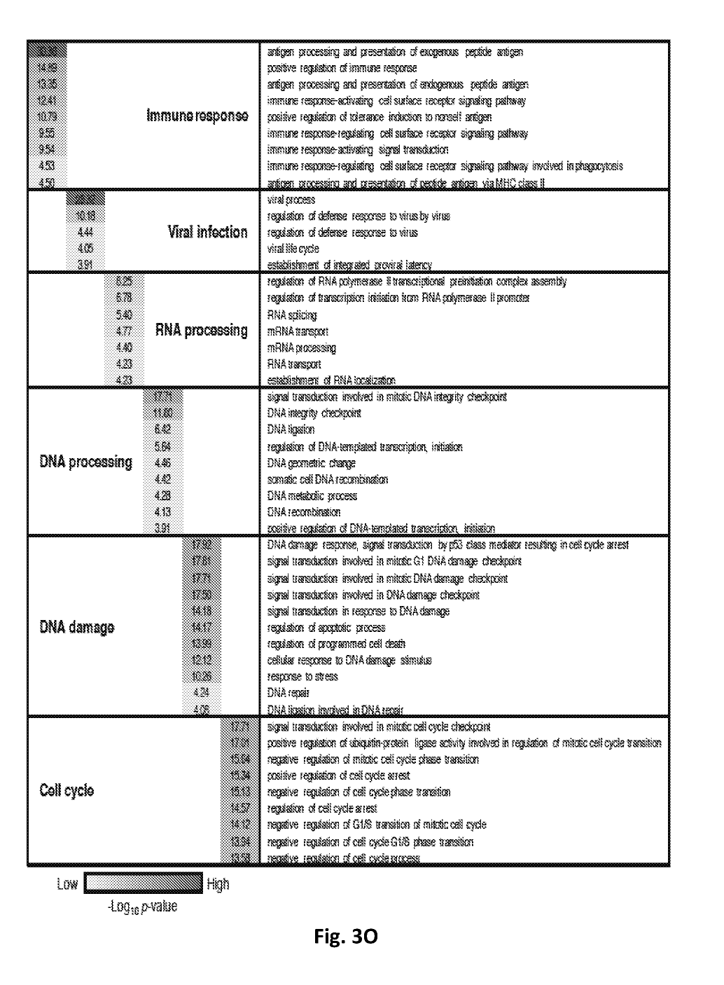

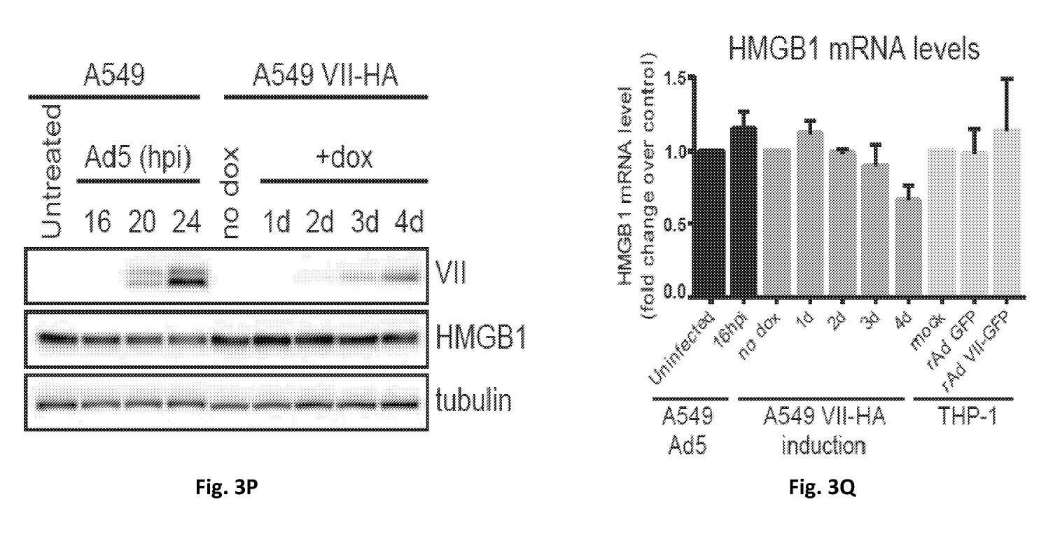

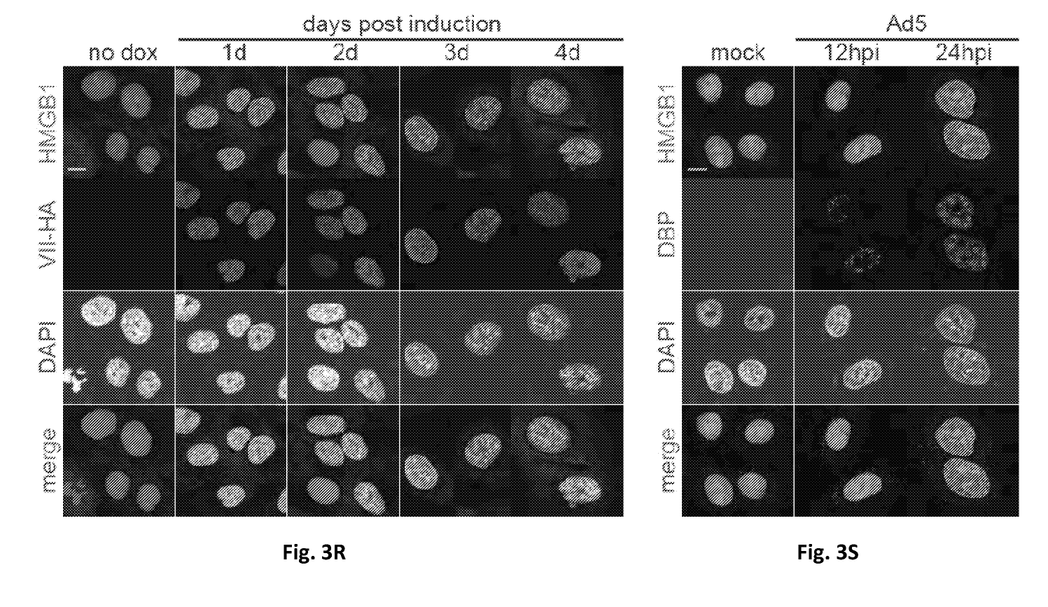

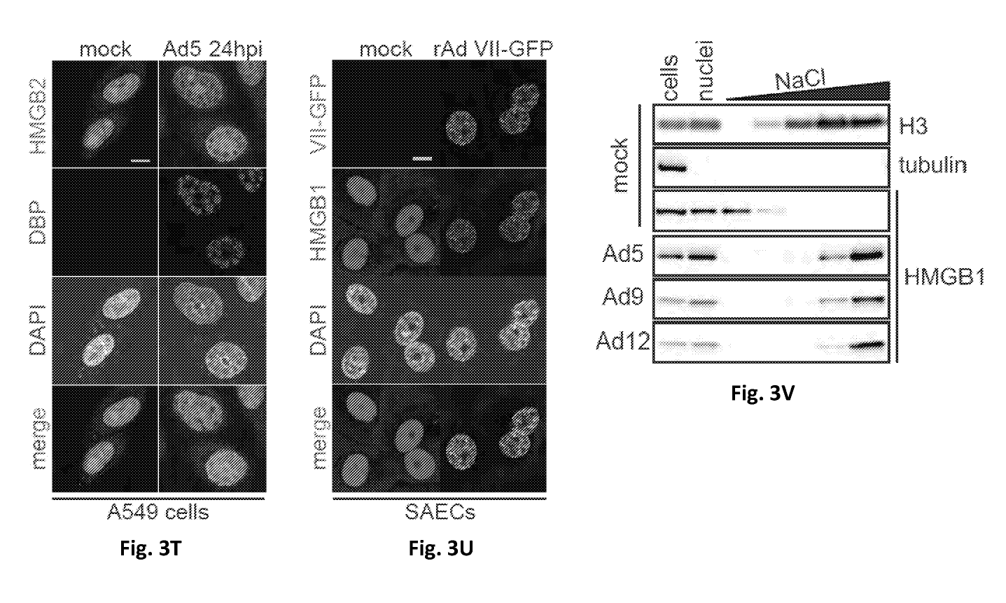

To determine whether protein VII manipulation of cellular chromatin is part of a strategy to counteract host defenses, mass spectrometry was employed to examine changes in protein composition of nuclear fractions. The total chromatin proteome in the presence and absence of protein VII was compared (FIG. 3A). 20 proteins were identified that changed significantly across three biological replicates (FIGS. 3L, 3M, 3N, 30 and Table 2). The categories of proteins most significantly changed upon protein VII expression were related to immune responses (FIG. 3N). The top four proteins enriched in chromatin fractions by protein VII were SET/TAF-1, a protein previously shown to interact with protein VII (Gyurcsik et al. (2006) Biochem., 45:303-313; Haruki et al. (2006) J. Virol., 80:794-801), and high mobility group box proteins HMGB1, HMGB2 and HMGB3 (FIG. 3A). The HMGB proteins are alarmins with multiple functions as activators of immunity and inflammation (Kang et al. (2014) Mol. Aspects Med., 40:1-116; Lotze et al. (2005) Nat. Rev. Immunol., 5:331-342). HMGB1 is a nuclear protein normally only transiently associated with chromatin (Scaffidi et al. (2002) Nature 418:191-195; Sapojnikova et al. (2005) Biochim. Biophys. Acta 1729:57-63). Cells also release HMGB1 as an extracellular danger signal that promotes immune responses after injury or infection (Lu et al. (2012) Nature 488:670-674). Increased chromatin association of HMGB1 and HMGB2 was confirmed by analysis of fractionated nuclei, upon protein VII expression and during adenovirus infection (FIG. 3B). These changes are not due to altered HMGB1 expression levels (FIGS. 3P, 3Q). Direct binding of recombinant protein VII to HMGB1 was demonstrated in vitro and HMGB1 co-immunopreciptation with protein VII was confirmed (FIG. 3C). Reorganization of HMGB1 and HMGB2 distribution was observed upon protein VII expression, and at late stages of infection (FIGS. 3D-3F, FIGS. 3R-3T). Reorganization of HMGB1 distribution was also shown by vector transduction to express protein VII-GFP (FIGS. 3G, 3U). The effect of protein VII on HMGB1 is also conserved across human Ad serotypes (FIG. 3V). Effects of protein VII on HMGB1 mobility were defined by fluorescence recovery after photobleaching (FRAP) and decreased HMGB1 diffusion was found (FIG. 3H). It was next investigated whether protein VII is necessary for chromatin retention of HMGB1 during virus infection. A replication competent adenovirus with loxP sites inserted on either side of the protein VII gene was used, allowing deletion of protein VII during infection of cells expressing Cre recombinase (FIGS. 31, 3J, 4G, 4H). Nuclei from infected cells were fractionated and HMGB1 and HMGB2 were no longer retained in chromatin when protein VII was deleted (FIG. 3K, 4I). Together, these data indicate that protein VII is necessary and sufficient to promote chromatin association and immobilization of HMGB1.

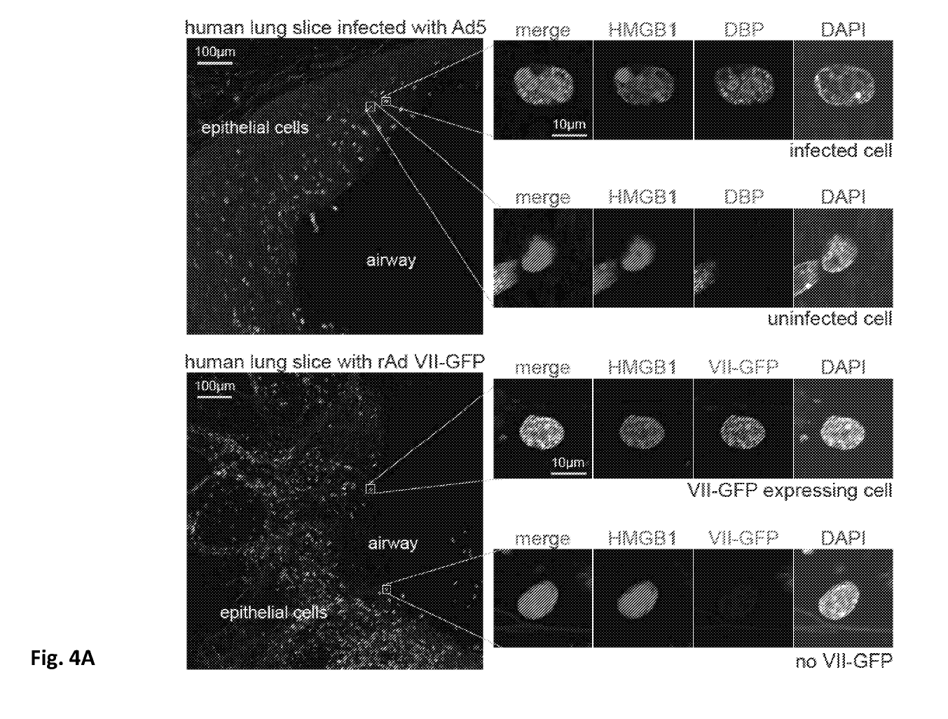

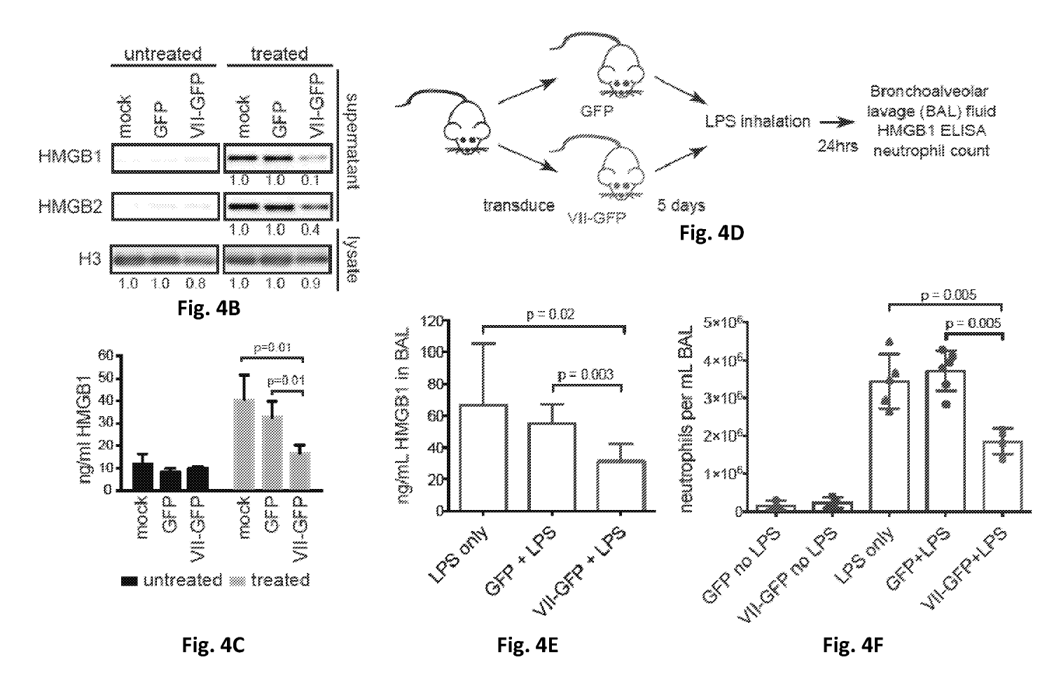

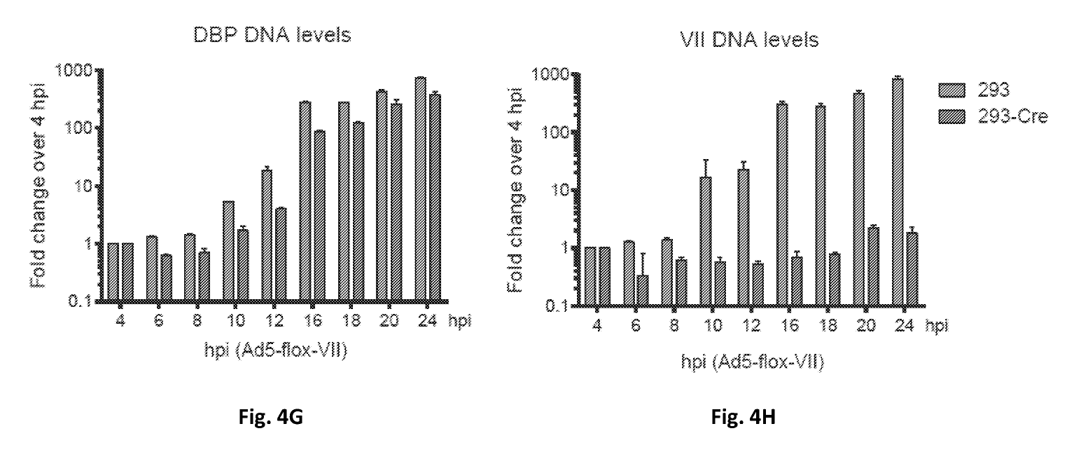

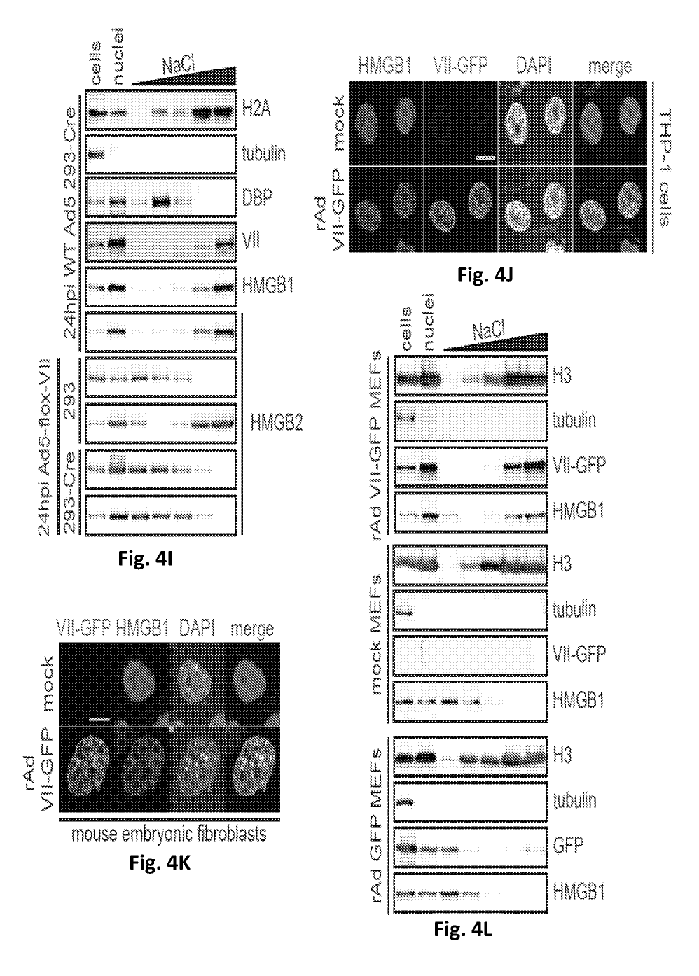

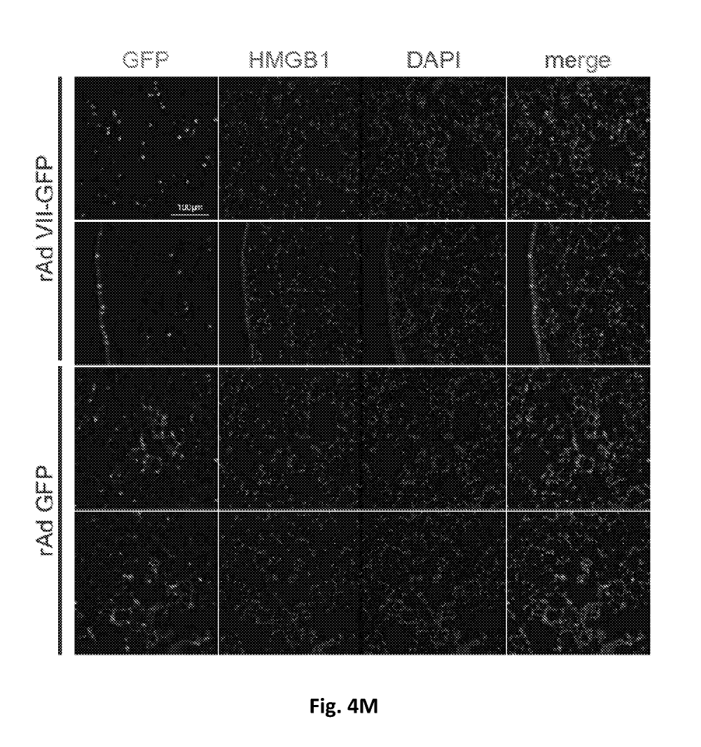

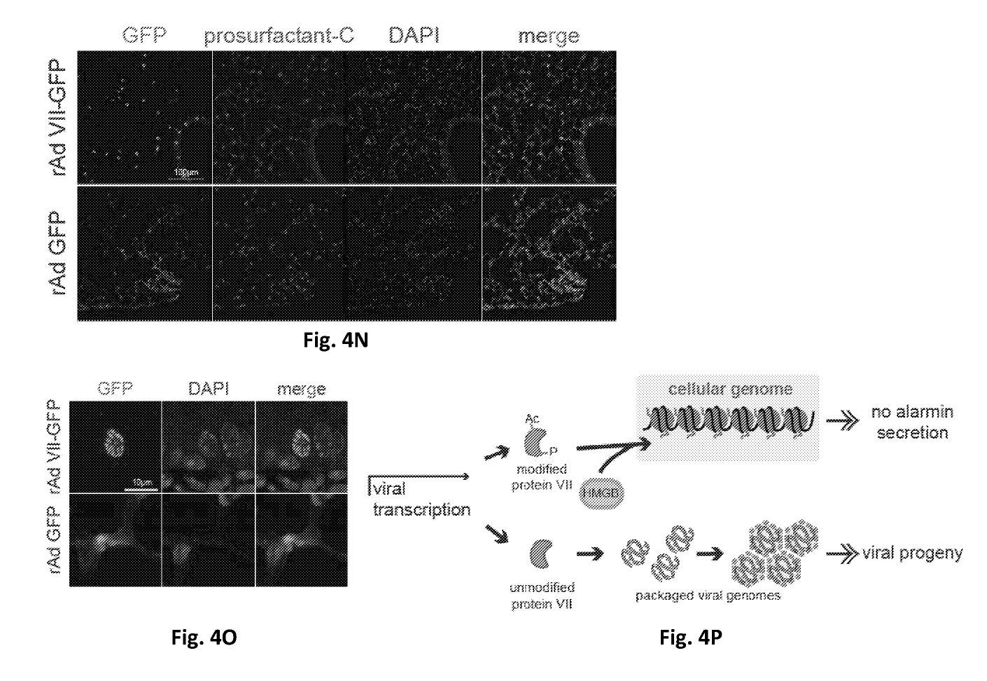

It was hypothesized that protein VII retains HMGB1 in chromatin during natural infection to prevent cellular release and abrogate host immune responses. Endogenous HMGB1 was visualized during adenovirus infection in precision cut lung slices (PCLS) (Koziol-White et al. (2011) Exp. Rev. Respir. Med., 5:767-777) from human donors (FIG. 4A). Consistent with cell culture experiments, protein VII is sufficient to relocalize endogenous HMGB1. It was then tested whether protein VII prevents HMGB1 release in cell culture and in vivo models. GFP or protein VII-GFP was expressed in macrophage-like THP-1 cells, and protein VII-GFP was confirmed to be sufficient to alter chromatin and HMGB1 localization (FIG. 4J). Cells were treated to stimulate inflammasomes, and HMGB1 analyzed in supernatants. Protein VII expression resulted in reduced levels of HMGB1 and HMGB2 in supernatants (FIGS. 4B, 4C). Subsequently, a murine model of LPS induced lung injury (Ueno et al. (2004) Am. J. Respir. Crit. Care Med., 170:1310-1316) was employed to investigate protein VII's impact on HMGB1 release and neutrophil recruitment in vivo (FIG. 4D). Protein VII was confirmed to be expressed in transduced mouse lungs (FIGS. 4M-40) and retained mouse HMGB1 (FIGS. 4K, 4L). Mice inhaled LPS to induce HMGB1 release and neutrophil recruitment to alveoli. Bronchoalveolar lavage (BAL) fluid obtained 24 hours after LPS exposure showed that mice transduced to express protein VII had significantly less HMGB1 and fewer neutrophils than mice expressing GFP (FIGS. 4D-4F). Together, these data indicate that protein VII functions in cellular chromatin to retain HMGB1 as a mechanism to blunt immune responses.

In summary, in addition to known roles on packaged viral DNA (Johnson et al. (2004) J. Virol., 78:6459-6468; Karen et al. (2011) J. Virol., 85:4135-4142), it is shown that protein VII interacts with cellular chromatin and binds nucleosomes. Protein VII PTMs contribute to chromatin localization, and that protein VII impacts chromatin-association of host proteins. Finally, protein VII in cellular chromatin leads to sequestration of HMGB family members, contributing to abrogated immune responses (FIG. 4P). The study reveals that chromatin retention of signaling molecules by a viral protein represents a previously unrecognized immune evasion strategy.

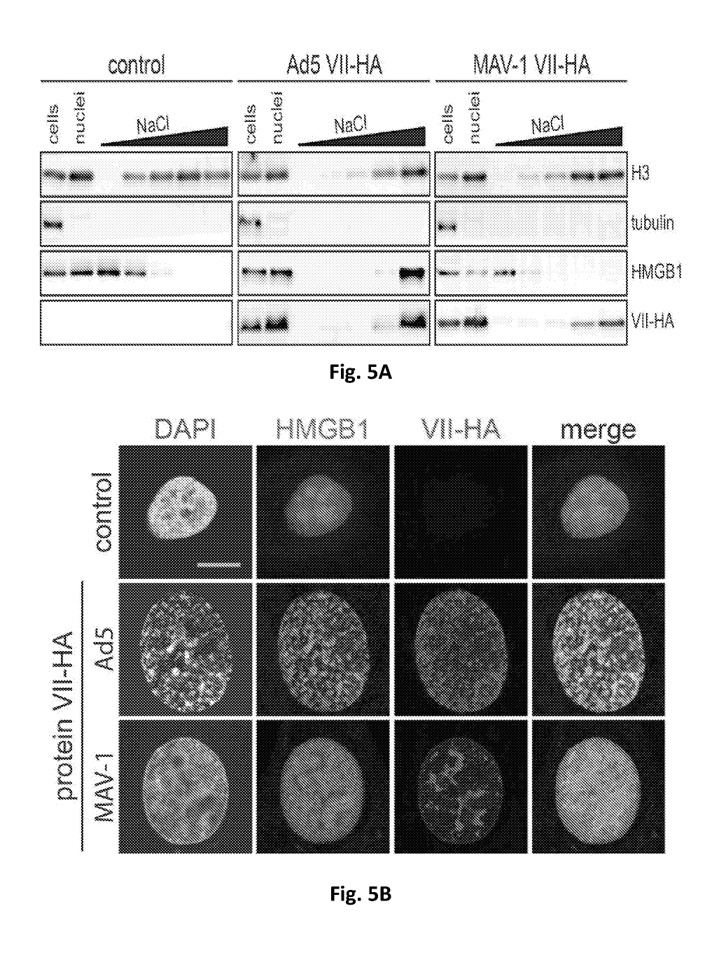

In additional experiments, data was generated demonstrating that human, but not mouse adenovirus protein VII retains HMGB1 in chromatin. FIG. 5A shows a western blot of chromatin fractionation of A549 cells uninduced (control) and induced for expression of protein VII-HA from human adenovirus serotype 5 (Ad5 VII-HA) or protein VII-HA from mouse adenovirus serotype 1 (MAV-1 VII-HA) by doxycycline treatment for 4 days. HMGB1 elutes under low salt conditions in control conditions. Expression of Ad5 protein VII results in retention in the high salt chromatin fractions while MAV-1 protein VII does not effect the elution of HMGB1.

Immunofluorescence of HMGB1 in A549s cells under control conditions or upon expression of Ad5 or MAV-1 protein VII-HA is provided in FIG. 5B. HMGB1 has a pan-nuclear staining in control cells. Expression of Ad5 protein VII but not MAV-1 protein VII results in relocalization of HMGB1 into the same pattern as the viral protein.

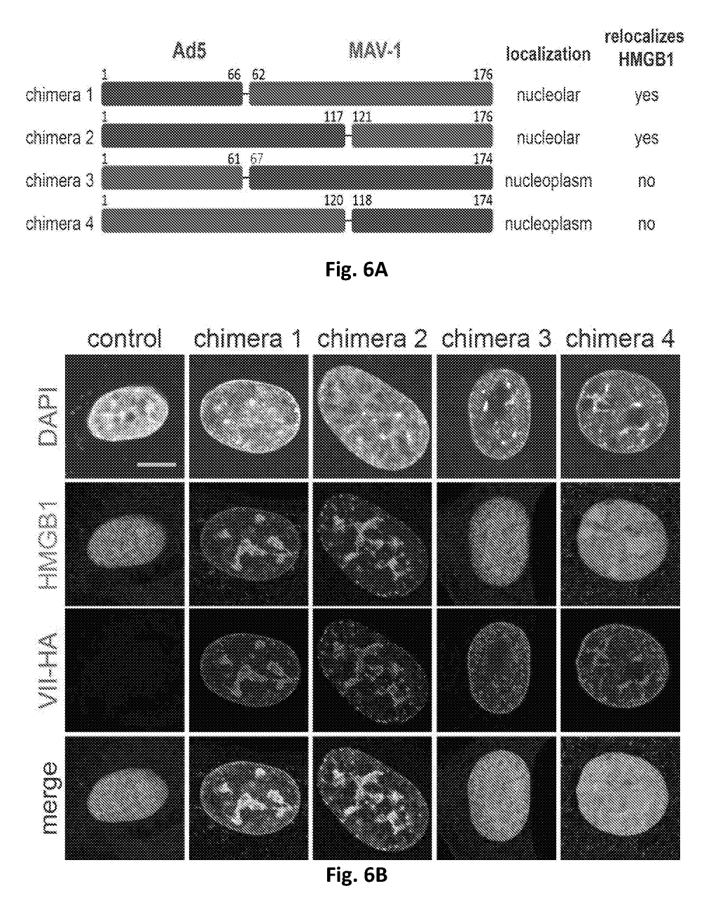

These data show that the interaction of protein VII with HMGB1 is not conserved in mouse adenovirus. This difference between the Ad5 and MAV-1 protein VII allowed us to map the HMGB1-binding region through expression of protein VII chimeras. Our results reveal that the first 66 amino acids of human adenovirus protein VII are effective to relocalize HMGB1. FIG. 6A shows a schematic diagram of the different chimeras generated between Ad5 and MAV-1 protein VII indicating the regions of each protein present in the construct, the localization within the cell and the ability to relocalize HMGB1. Immunofluorescence of HMGB1 in A549s cells under control conditions or upon expression the different protein VII chimeras is shown in FIG. 6B. Expression of chimeras 1 and 2 containing the first 66 amino acids of Ad5 protein VII relocalizes HMGB1 into the same pattern as the chimera.

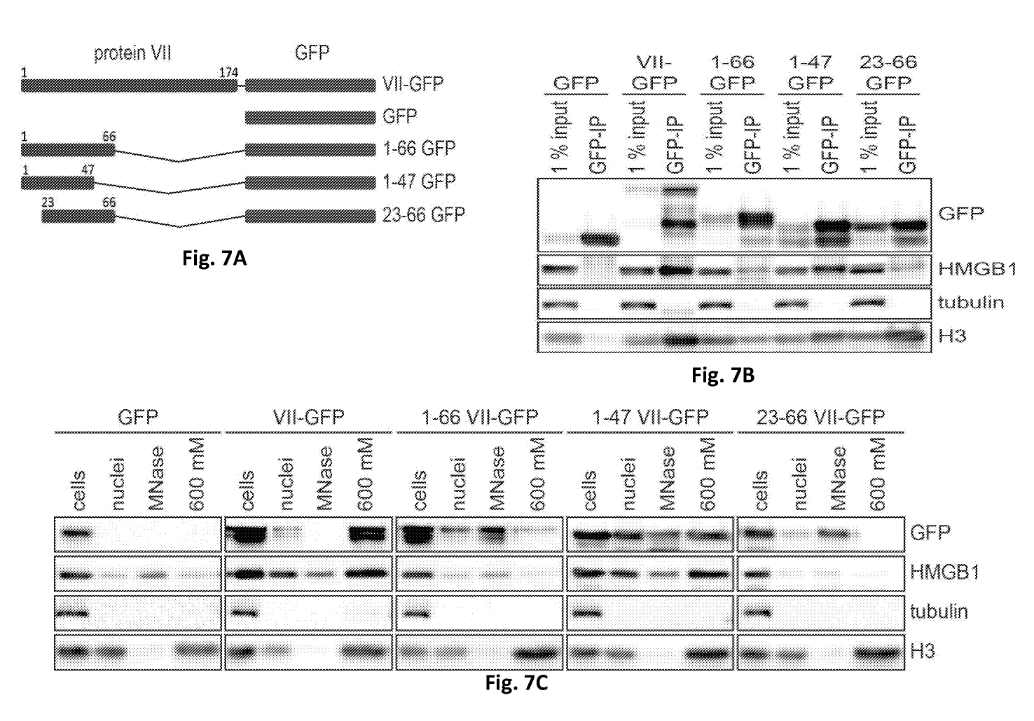

These data indicate that the first 66 amino acids of Ad5 protein VII contain the HMGB1-binding region. We further narrowed down this binding region through expression of protein VII peptides tagged with GFP. These results indicated that the first 47 amino acids of human adenovirus protein VII bind HMGB1 in cells. FIG. 7A shows a schematic diagram of the different ADS protein VII constructs tagged with GFP assessed. FIG. 7B shows the results from GFP immunoprecipitation from A549 cells transfected for 24 hours with GFP or the different protein VII-GFP constructs. Full-length protein VII and the 1-66 as well as the 1-47 constructs are able to pull-down HMGB1 in these assays. Cellular fractionation of A549 cells transfected for 24 hours with GFP and the different protein VII constructs is shown in FIG. 7C. Expression of full-length and the 1-47 protein VII construct result in retention of HMGB1 in the 600 mM high-salt fraction.

These data demonstrate that the first 47 amino acids of Ad5 protein VII are sufficient to bind and relocalize HMGB1 in cells. Next, we mapped the region of HMGB1 bound by protein VII in cells and in vitro.

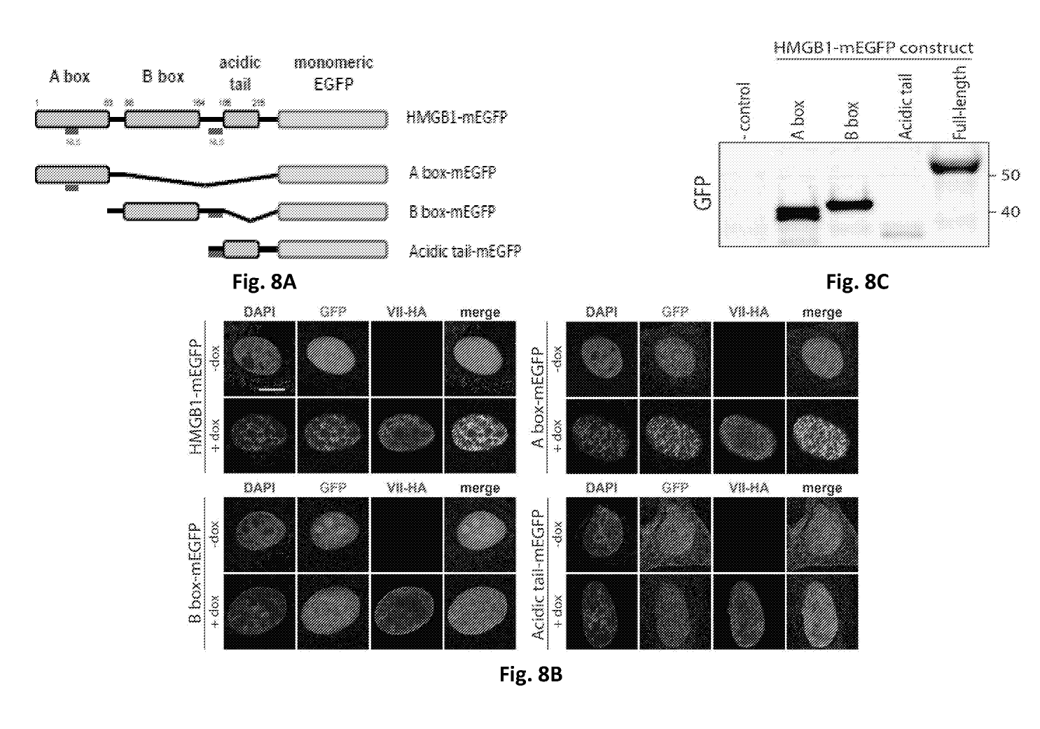

These results showed that protein VII relocalizes the HMGB1 A box, but not the B box or acidic tail. FIG. 8A provides a schematic of the different GFP constructs expressing distinct HMGB1 domains. As shown in FIG. 8A, HMGB1 contains 3 distinct domains: two DNA-binding domains called A box and B box and a highly acidic tail. Immunofluorescence of the HMGB1-GFP constructs 2 days after transfection of A549s cells under control conditions or upon expression of Ad5 protein VII-HA is shown in FIG. 8B. FIG. 8C shows the results obtained from western blotting to assess expression levels of the HMGB1-GFP constructs. Only full-length HMGB1 and the HMGB1 A box are relocalized upon expression of protein VII. These data indicate that protein VII and biologically active fragments thereof are able to bind and relocalize the HMGB1 A box in cells. Next we tested the immobilization of this construct in chromatin upon protein VII expression.

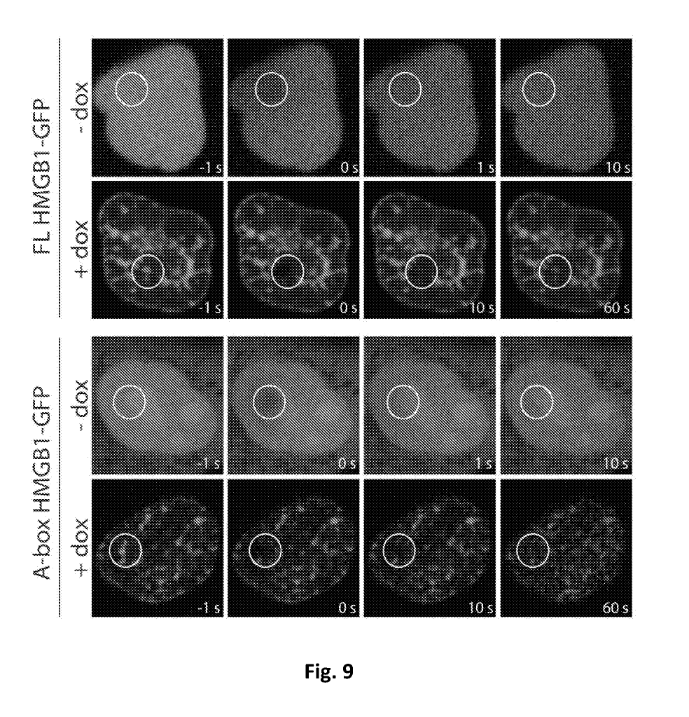

The results provided in FIG. 9 show that protein VII immobilizes HMGB1 A box in chromatin similar to that observed with the full length HMGB1 protein. FIG. 9 is a photograph depicting an immunofluorescence time course showing the fluorescence recovery after photo-bleaching of full-length HMGB1 or just the HMGB1 A box. Under control conditions the fluorescence of the bleached area (white circle) recovers within seconds for both constructs. Upon expression of protein VII the recovery is dramatically delayed, indicating that both the full-length HMGB1 and the HMGB1 A box are immobilized.

These data indicate that the interaction between protein VII and the HMGB1 A box is strong enough to immobilize the HMGB1 in chromatin. Next we tested whether protein VII also interacts with the HMGB1 A box in vitro.

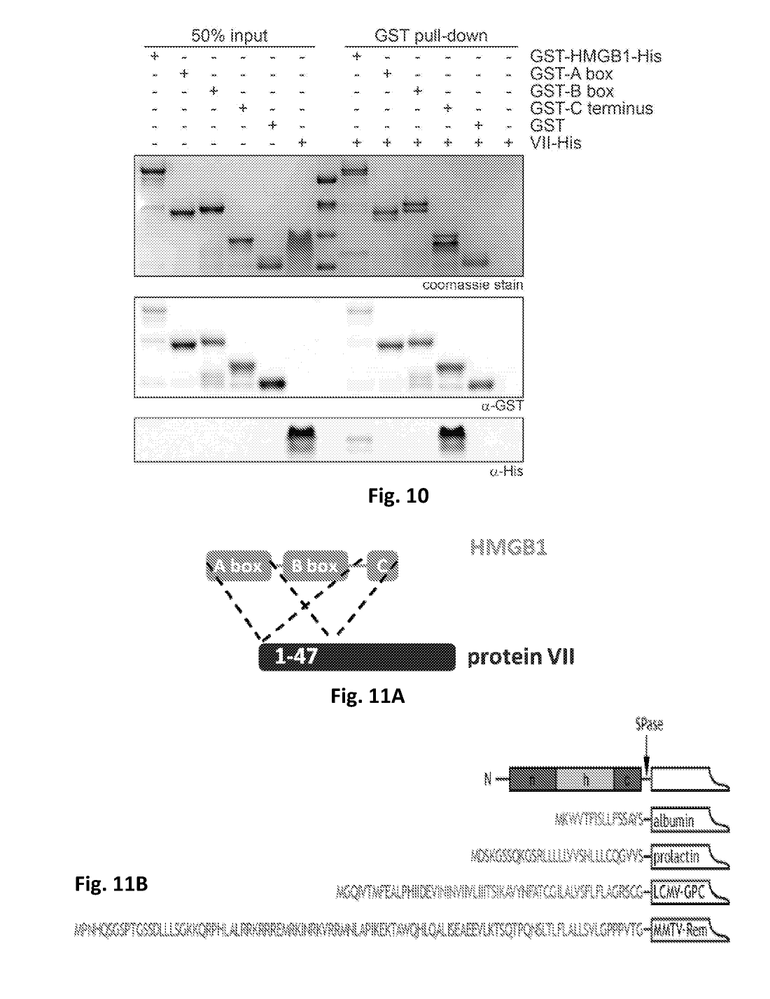

Our results show that protein VII binds to the HMGB1 acidic tail in vitro. FIG. 10 shows an in vitro GST pull down of recombinant GST constructs encoding full-length HMGB1 or distinct HMGB1 domains shown as coomassie stained SDS-PAGE gel or Western blot for the GST or His. Only the full-length HMGB1 construct and the HMGB1 C-terminus were able to also pull-down protein VII. These data indicate that in vitro protein VII directly interacts with the HMGB1 highly acidic C-terminus. Together with the immunofluorescence data, these data demonstrate that protein VII is able to bind both the HGMB1 A box as well as the acidic tail.

The first 47 amino acids of protein VII bind both the HMGB1 A box as well as the acidic tail in a 3D structure, and cover the immune receptor binding sites present in HMGB1. This finding provides new avenues for impacting inflammatory pathways which are modulated by HMGB1 activity via the use of biologically active protein VII fragments used alone or in combination with anti inflammatory agents.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An isolated protein VII peptide from a human adenovirus, wherein said peptide is less than about 80 amino acids in length.

2. The isolated protein VII peptide of claim 1, wherein said peptide is between 66 and 45 amino acids in length inclusive of the N-terminus of protein VII and further comprises a tag sequence.

3. The isolated protein VII peptide of claim 1, wherein said protein VII is from adenovirus type 5 and is 47 amino acids in length.

4. The isolated protein VII peptide of claim 1, wherein said peptide is acetylated.

5. The isolated protein VII peptide of claim 1, wherein said N-terminus is blocked.

6. The isolated protein VII peptide of claim 2, wherein said tag is selected from the group consisting of FLAG, HA, biotin, and His.

7. The isolated protein VII peptide of claim 6, wherein said tag is a FLAG tag having a sequence of DYKDDDDK.

8. The protein VII peptide of claim 1, wherein said peptide comprises amino acids 23-66 of protein VII.

9. A composition comprising at least one protein VII peptide of claim 1 and at least one carrier and optionally an anti-inflammatory agent.

10. A composition comprising the peptide of claim 1, at least one anti-inflammatory agent and a carrier.

11. An isolated nucleic acid encoding a protein VII peptide from a human adenovirus consisting of amino acids 1-47, 1-66, or 1-80, operably linked to signal peptide sequence.

12. An expression vector comprising the isolated nucleic acid of claim 11.

13. The expression vector of claim 12 selected from the group consisting of an adenoviral vector, an adeno-associated viral vector, a lentiviral vector, a plasmid vector, a herpes simplex virus vectors, and a vaccinia virus vector.

14. The isolated nucleic acid of claim 11, wherein said signal peptide is selected from SEQ ID NOS: 27-30.

15. A method for reducing, inhibiting, and/or preventing inflammation in a subject, said method comprising administering a protein VII peptide of claim 1 to said subject.

16. The method of claim 15, wherein said protein VII peptide comprises a FLAG tag.

17. The method of claim 15, further comprising administering at least one anti-inflammatory agent to said subject.

18. The method of claim 15, wherein said peptide is infused in to said patient.

19. The method of claim 15, wherein said peptide is aerosolized form and administered via an inhaler.

20. A method for treating, inhibiting, and/or preventing an inflammatory disease or disorder in a subject, said method comprising administering a protein VII peptide as claimed in claim 1 to said subject.

21. The method of claim 20, wherein said protein VII peptide is from adenovirus type 5 and is 47 amino acids in length.

22. The method of claim 20, further comprising administering at least one anti-inflammatory agent to said subject.

23. The method of claim 20, wherein said inflammatory disease or disorder is selected from the group consisting of arthritis, sepsis, ARDS, organ failure, ischemia, cancer, infection, colitis, trauma, endotoxemia, sickle cell acute chest syndrome, severe pneumonia, and respiratory tract inflammation.

24. The method of claim 20, wherein said disorder is ARDS and said peptide is administered in aerosolized form.

25. The method of claim 20, wherein said disorder is COPD and said peptide is administered in aerosolized form.
